(19) United States
(12) Patent Application Publication
(10) Pub. No.: US 2025/0204836 A1
(43) Pub. Date: Jun. 26, 2025

(54) Processing Short Term ECG-Based Noise Artifacts in Wearable Cardiac Systems

(71) Applicant: ZOLL Medical Corporation, Chelmsford, MA (US)

(72) Inventors: Jeffery T. Mitchell, Mars, PA (US); Michael A. Sipe, Pittsburgh, PA (US)

(21) Appl. No.: 18/989,059

(22) Filed: Dec. 20, 2024

Related U.S. Application Data
(60) Provisional application No. 63/613,824, filed on Dec. 22, 2023.

(51) Int. Cl. A61B 5/364; A61B 5/00; A61B 5/256; A61B 5/257; A61N 1/04

(52) U.S. Cl. CPC A61B 5/364; A61B 5/0006; A61B 5/256; A61B 5/257; A61N 1/046

(57) ABSTRACT

A cardiac event monitoring system for removing short-term noise artifacts from an electrocardiogram (ECG) signal includes a wearable cardiac sensing device having a plurality of physiological sensors; and a non-transitory memory configured to store ECG data. A processor in communication with the wearable cardiac sensing device is configured to receive the ECG data; obtain first order ECG data by removing first order artifacts; determine representative ECG data point amplitudes for predetermined time windows applied to the first order ECG data; determine a slope between substantially adjacent representative ECG amplitude data points; compare the slope to a slope threshold; obtain second order ECG data by identifying segments of the first order ECG data corresponding to the representative ECG amplitude data points having a slope that transgresses the slope threshold; process the second order ECG data; and determine whether an abnormal cardiac event has occurred based on the processed ECG data.

### CROSS REFERENCE TO RELATED APPLICATIONS

This application claims the benefit of U.S. Provisional Patent Application No. 63/613,824, titled “PROCESSING SHORT TERM ECG-BASED NOISE ARTIFACTS IN WEARABLE CARDIAC SYSTEMS,” filed Dec. 22, 2023, the disclosure of which is hereby incorporated in its entirety by reference.

### TECHNICAL FIELD

The present disclosure relates to a wearable cardiac sensing system configured to detect and compensate for short-term noise artifacts in ECG signals.

### BACKGROUND

There are a wide variety of electronic and mechanical devices for monitoring and treating patients' medical conditions. In some examples, depending on the underlying medical condition being monitored or treated, medical devices such as cardiac monitors or defibrillators may be surgically implanted or externally connected to the patient. In some cases, physicians may use medical devices alone or in combination with drug therapies to treat conditions such as cardiac arrhythmias. A patient with a known cardiac condition may be provided with a device that monitors the patient's cardiac activity. The device or a remote server in communication with the device may analyze the patient's cardiac activity as part of monitoring the patient's ongoing cardiac health.

### SUMMARY

In one or more examples, a cardiac event monitoring system for removing short-term noise artifacts from an electrocardiogram (ECG) signal is provided. The system includes a wearable cardiac sensing device configured to be bodily-attached to the patient. The wearable cardiac sensing device comprises a plurality of physiological sensors comprising one or more ECG electrodes configured to sense electrical signals from a skin surface of the patient; and a non-transitory memory configured to store ECG data based on the electrical signals of the patient. The system also includes a processor in communication with the wearable cardiac sensing device. The processor is configured to receive the ECG data from the memory; obtain first order ECG data by processing the ECG data to remove first order artifacts; determine representative ECG data point amplitudes for predetermined time windows applied to the first order ECG data; determine a slope between substantially adjacent representative ECG amplitude data points; compare the slope to a slope threshold indicative of a presence of a second order short-term noise artifact; obtain second order ECG data by identifying segments of the first order ECG data corresponding to the representative ECG amplitude data points having a slope that transgresses the slope threshold; process the second order ECG data to compensate for the second order short-term noise artifacts to obtain processed ECG data; and determine whether an abnormal cardiac event has occurred based on the processed ECG data.

Implementations of the cardiac event monitoring system can include one or more of the following features. The second order ECG data may be processed by removing the segments of the first order ECG data corresponding to the representative ECG amplitude data points having a slope that transgresses the slope threshold. In other examples, the second order ECG data may be processed by averaging segments of the ECG data that neighbor the first order ECG data corresponding to the representative ECG amplitude data points having a slope that transgresses the slope threshold and replacing the first order ECG data corresponding to the representative ECG amplitude data points having a slope that transgresses the slope threshold with the average. In some examples, the second order ECG data may be processed by determining a noise score for the first order ECG data corresponding to the representative ECG amplitude data points having a slope that transgresses the slope threshold.

In some examples, the second order short-term noise artifacts may comprise at least one of a motion artifact, electrostatic interference, triboelectric interference, and electromagnetic interference. The processor may be further configured obtain at least two ECG signal channels from the plurality of physiological sensors. The at least two ECG signal channels may comprise a primary ECG signal channel and a secondary ECG signal channel. The secondary ECG channel may be provided to validate the presence of the second order short-term noise artifact.

In other examples, the predetermined time windows may have a duration of at least one of 1 ms, 10 ms, 100 ms, or 1000 ms. The short-term noise artifact may be a motion artifact that lasts for a duration of at least one of about 0.1 ms, 10 ms, 100 ms, 1 second, 2 seconds, 5 seconds, or 10 seconds. The slope threshold may be at least one of 2, 3, 5, or 7 and may be determined based on a distribution of the determined slopes of the first order ECG data. In some examples, the representative ECG data point amplitudes may be an average of amplitudes of the first order ECG data within the predetermined time windows. The average of the amplitudes of the first order ECG data may be obtained using ensemble averaging.

The wearable cardiac sensing device may further include a garment configured to be worn around the patient's torso for an extended period of time. At least a portion of the plurality of physiological sensors may be configured to be removably mounted onto the garment. At least a portion of the plurality of physiological sensors may be permanently integrated into the garment. The plurality of physiological sensors may include at least three physiological sensors and the garment may further include one or more therapy electrodes incorporated therein. The one or more therapy electrodes may be positioned within the garment to deliver one or more therapeutic defibrillating shocks to the patient.

The wearable cardiac sensing device may include a removable adhesive patch configured to be adhered to skin of the patient. At least a portion of the plurality of physiological sensors may be configured to be removably mounted onto the removable adhesive patch. The wearable cardiac sensing device may further comprise a cardiac sensing unit incorporating the portion of the plurality of physiological sensors and the cardiac sensing unit may be configured to be removably mounted onto the removable adhesive patch.

In some examples, the wearable cardiac sensing device may further comprise at least one tissue fluid monitor configured to measure a fluid level in tissue of the patient. The at least one tissue fluid monitor may comprise one or more antennas configured to direct RF waves through the tissue of the patient and measure output RF signals in response to the RF waves that have passed through the tissue. The output RF signals may comprise parameters indicative of the fluid level in the tissue of the patient.

The wearable cardiac sensing device may comprise the processor. In some examples, the wearable cardiac sensing device may be in communication with a remote server, and the processor may be further configured to transmit at least one of the processed ECG data or an indication of the abnormal cardiac event to the remote server.

In one or more examples, a non-transitory computer-readable medium storing sequences of instructions executable by at least one processor is provided. The sequences of instructions instruct the at least one processor to: receive ECG data; obtain first order ECG data by processing the ECG data to remove first order artifacts; determine representative ECG data point amplitudes for predetermined time windows applied to the first order ECG data; determine a slope between substantially adjacent representative ECG amplitude data points; compare the slope to a slope threshold indicative of a presence of a second order short-term noise artifact; obtain second order ECG data by identifying segments of the first order ECG data corresponding to the representative ECG amplitude data points having a slope that transgresses the slope threshold; process the second order ECG data to compensate for the second order short-term noise artifacts to obtain processed ECG data; and determine whether an abnormal cardiac event has occurred based on the processed ECG data.

In one or more examples, a method for removing short-term noise artifacts from an electrocardiogram (ECG) signal is provided. The method comprises: receiving ECG data; obtaining first order ECG data by processing the ECG data to remove first order artifacts; determining representative ECG data point amplitudes for predetermined time windows applied to the first order ECG data; determining a slope between substantially adjacent representative ECG amplitude data points; comparing the slope to a slope threshold indicative of a presence of a second order short-term noise artifact; obtaining second order ECG data by identifying segments of the first order ECG data corresponding to the representative ECG amplitude data points having a slope that transgresses the slope threshold; processing the second order ECG data to compensate for the second order short-term noise artifacts to obtain processed ECG data; and determining whether an abnormal cardiac event has occurred based on the processed ECG data.

In one or more examples, a cardiac event monitoring system for removing short-term noise artifacts from an electrocardiogram (ECG) signal is provided. The system comprises a processor in communication with a wearable cardiac sensing device. The processor is configured to receive ECG data based on electrical signal from a skin surface of a patient obtained from a plurality of physiological sensors of the wearable cardiac sensing device; obtain first order ECG data by processing the ECG data to remove first order artifacts; determine representative ECG data point amplitudes for predetermined time windows applied to the first order ECG data; determine a slope between substantially adjacent representative ECG amplitude data points; compare the slope to a slope threshold indicative of a presence of a second order short-term noise artifact; obtain second order ECG data by identifying segments of the first order ECG data corresponding to the representative ECG amplitude data points having a slope that transgresses the slope threshold; process the second order ECG data to compensate for the second order short-term noise artifacts to obtain processed ECG data; and determine whether an abnormal cardiac event has occurred based on the processed ECG data.

Preferred and non-limiting examples of the present disclosure will now be described in the following numbered clauses:

Clause 1: A cardiac event monitoring system for removing short-term noise artifacts from an electrocardiogram (ECG) signal, comprising: a wearable cardiac sensing device configured to be bodily-attached to the patient, the wearable cardiac sensing device comprising a plurality of physiological sensors comprising one or more ECG electrodes configured to sense electrical signals from a skin surface of the patient; and a non-transitory memory configured to store ECG data based on the electrical signals of the patient; and a processor in communication with the wearable cardiac sensing device, the processor configured to receive the ECG data from the memory; obtain first order ECG data by processing the ECG data to remove first order artifacts; determine representative ECG data point amplitudes for predetermined time windows applied to the first order ECG data; determine a slope between substantially adjacent representative ECG amplitude data points; compare the slope to a slope threshold indicative of a presence of a second order short-term noise artifact; obtain second order ECG data by identifying segments of the first order ECG data corresponding to the representative ECG amplitude data points having a slope that transgresses the slope threshold; process the second order ECG data to compensate for the second order short-term noise artifacts to obtain processed ECG data; and determine whether an abnormal cardiac event has occurred based on the processed ECG data.

Clause 2: The cardiac event monitoring system of clause 1, wherein the second order ECG data is processed by removing the segments of the first order ECG data corresponding to the representative ECG amplitude data points having a slope that transgresses the slope threshold.

Clause 3: The cardiac event monitoring system of clause 1 or clause 2, wherein the second order ECG data is processed by averaging segments of the ECG data that neighbor the first order ECG data corresponding to the representative ECG amplitude data points having a slope that transgresses the slope threshold and replacing the first order ECG data corresponding to the representative ECG amplitude data points having a slope that transgresses the slope threshold with the average.

Clause 4: The cardiac event monitoring system of any of clauses 1-3, wherein the second order ECG data is processed by determining a noise score for the first order ECG data corresponding to the representative ECG amplitude data points having a slope that transgresses the slope threshold.

Clause 5: The cardiac event monitoring system of any of clauses 1-4, wherein the second order short-term noise artifacts comprise at least one of a motion artifact, electrostatic interference, triboelectric interference, and electromagnetic interference.

Clause 6: The cardiac event monitoring system of any of clauses 1-5, wherein the processor is further configured obtain at least two ECG signal channels from the plurality of physiological sensors.

Clause 7: The cardiac event monitoring system of clause 6, wherein the at least two ECG signal channels comprise a primary ECG signal channel and a secondary ECG signal channel.

Clause 8: The cardiac event monitoring system of clause 7, wherein the secondary ECG channel is provided to validate the presence of the second order short-term noise artifact.

Clause 9: The cardiac event monitoring system of any of clauses 1-8, wherein the predetermined time windows have a duration of at least one of 1 ms, 10 ms, 100 ms, or 1000 ms.

Clause 10: The cardiac event monitoring system of any of clauses 1-9, wherein the short-term noise artifact is a motion artifact that lasts for a duration of at least one of about 0.1 ms, 10 ms, 100 ms, 1 second, 2 seconds, 5 seconds, or 10 seconds.

Clause 11: The cardiac event monitoring system of any of clauses 1-10, wherein the slope threshold is at least one of 2, 3, 5, or 7.

Clause 12: The cardiac event monitoring system of any of clauses 1-11, wherein the slope threshold is determined based on a distribution of the determined slopes of the first order ECG data.

Clause 13: The cardiac event monitoring system of any of clauses 1-12, wherein the representative ECG data point amplitudes are an average of amplitudes of the first order ECG data within the predetermined time windows.

Clause 14: The cardiac event monitoring system of clause 13, wherein the average of the amplitudes of the first order ECG data is obtained using ensemble averaging.

Clause 15: The cardiac event monitoring system of any of clauses 1-14, wherein the wearable cardiac sensing device further comprises a garment configured to be worn around the patient's torso for an extended period of time.

Clause 16: The cardiac event monitoring system of clause 15, wherein at least a portion of the plurality of physiological sensors are configured to be removably mounted onto the garment.

Clause 17: The cardiac event monitoring system of clause 15 or clause 16, wherein at least a portion of the plurality of physiological sensors are permanently integrated into the garment.

Clause 18: The cardiac event monitoring system of any of clauses 15-17, wherein the plurality of physiological sensors comprise at least three physiological sensors and the garment further includes one or more therapy electrodes incorporated therein, wherein the one or more therapy electrodes are positioned within the garment to deliver one or more therapeutic defibrillating shocks to the patient.

Clause 19: The cardiac event monitoring system of any of clauses 1-18, wherein the wearable cardiac sensing device further comprises a removable adhesive patch configured to be adhered to skin of the patient.

Clause 20: The cardiac event monitoring system of clause 19, wherein at least a portion of the plurality of physiological sensors are configured to be removably mounted onto the removable adhesive patch.

Clause 21: The cardiac event monitoring system of clause 19 or clause 20, wherein the wearable cardiac sensing device further comprises a cardiac sensing unit incorporating the portion of the plurality of physiological sensors, the cardiac sensing unit configured to be removably mounted onto the removable adhesive patch.

Clause 22: The cardiac event monitoring system of any of clauses 1-21, wherein the wearable cardiac sensing device further comprises at least one tissue fluid monitor configured to measure a fluid level in tissue of the patient.

Clause 23: The cardiac monitoring system of clause 22, wherein the at least one tissue fluid monitor comprises one or more antennas configured to direct RF waves through the tissue of the patient and measure output RF signals in response to the RF waves that have passed through the tissue.

Clause 24: The cardiac monitoring system of clause 23, wherein the output RF signals comprise parameters indicative of the fluid level in the tissue of the patient.

Clause 25: The cardiac event monitoring system of any of clauses 1-24, wherein the wearable cardiac sensing device comprises the processor.

Clause 26: The cardiac event monitoring system of clause 25, wherein the wearable cardiac sensing device is in communication with a remote server, and wherein the processor is further configured to transmit at least one of the processed ECG data or an indication of the abnormal cardiac event to the remote server.

Clause 27: A non-transitory computer-readable medium storing sequences of instructions executable by at least one processor, the sequences of instructions instructing the at least one processor to: receive ECG data; obtain first order ECG data by processing the ECG data to remove first order artifacts; determine representative ECG data point amplitudes for predetermined time windows applied to the first order ECG data; determine a slope between substantially adjacent representative ECG amplitude data points; compare the slope to a slope threshold indicative of a presence of a second order short-term noise artifact; obtain second order ECG data by identifying segments of the first order ECG data corresponding to the representative ECG amplitude data points having a slope that transgresses the slope threshold; process the second order ECG data to compensate for the second order short-term noise artifacts to obtain processed ECG data; and determine whether an abnormal cardiac event has occurred based on the processed ECG data.

Clause 28: The non-transitory computer-readable medium of clause 27, wherein the second order ECG data is processed by removing the segments of the first order ECG data corresponding to the representative ECG amplitude data points having a slope that transgresses the slope threshold.

Clause 29: The non-transitory computer-readable medium of clause 27 or clause 28, wherein the second order ECG data is processed by averaging segments of the ECG data that neighbor the first order ECG data corresponding to the representative ECG amplitude data points having a slope that transgresses the slope threshold and replacing the first order ECG data corresponding to the representative ECG amplitude data points having a slope that transgresses the slope threshold with the average.

Clause 30: The non-transitory computer-readable medium of any of clauses 27-29, wherein the second order ECG data is processed by determining a noise score for the first order ECG data corresponding to the representative ECG amplitude data points having a slope that transgresses the slope threshold.

Clause 31: The non-transitory computer-readable medium of any of clauses 27-30, wherein the second order short-term noise artifacts comprise at least one of a motion artifact, electrostatic interference, triboelectric interference, and electromagnetic interference.

Clause 32: The non-transitory computer-readable medium of any of clauses 27-31, wherein the processor is further configured obtain at least two ECG signal channels from the plurality of physiological sensors.

Clause 33: The non-transitory computer-readable medium of clause 32, wherein the at least two ECG signal channels comprise a primary ECG signal channel and a secondary ECG signal channel.

Clause 34: The non-transitory computer-readable medium of clause 33, wherein the secondary ECG channel is provided to validate the presence of the second order short-term noise artifact.

Clause 35: The non-transitory computer-readable medium of any of clauses 27-34, wherein the predetermined time windows have a duration of at least one of 1 ms, 10 ms, 100 ms, or 1000 ms.

Clause 36: The non-transitory computer-readable medium of any of clauses 27-35, wherein the short-term noise artifact is a motion artifact that lasts for a duration of at least one of about 0.1 ms, 10 ms, 100 ms, 1 second, 2 seconds, 5 seconds, or 10 seconds.

Clause 37: The non-transitory computer-readable medium of any of clauses 27-36, wherein the slope threshold is at least one of 2, 3, 5, or 7.

Clause 38: The non-transitory computer-readable medium of any of clauses 27-37, wherein the slope threshold is determined based on a distribution of the determined slopes of the first order ECG data.

Clause 39: The non-transitory computer-readable medium of any of clauses 27-38, wherein the representative ECG data point amplitudes are an average of amplitudes of the first order ECG data within the predetermined time windows.

Clause 40: The non-transitory computer-readable medium of any of clauses 27-39, wherein the average of the amplitudes of the first order ECG data is obtained using ensemble averaging.

Clause 41: A method for removing short-term noise artifacts from an electrocardiogram (ECG) signal, comprising: receiving ECG data; obtaining first order ECG data by processing the ECG data to remove first order artifacts; determining representative ECG data point amplitudes for predetermined time windows applied to the first order ECG data; determining a slope between substantially adjacent representative ECG amplitude data points; comparing the slope to a slope threshold indicative of a presence of a second order short-term noise artifact; obtaining second order ECG data by identifying segments of the first order ECG data corresponding to the representative ECG amplitude data points having a slope that transgresses the slope threshold; processing the second order ECG data to compensate for the second order short-term noise artifacts to obtain processed ECG data; and determining whether an abnormal cardiac event has occurred based on the processed ECG data.

Clause 42: The method of clause 41, wherein the second order ECG data is processed by removing the segments of the first order ECG data corresponding to the representative ECG amplitude data points having a slope that transgresses the slope threshold.

Clause 43: The method of clause 41 or clause 42, wherein the second order ECG data is processed by averaging segments of the ECG data that neighbor the first order ECG data corresponding to the representative ECG amplitude data points having a slope that transgresses the slope threshold and replacing the first order ECG data corresponding to the representative ECG amplitude data points having a slope that transgresses the slope threshold with the average.

Clause 44: The method of any of clauses 41-43, wherein the second order ECG data is processed by determining a noise score for the first order ECG data corresponding to the representative ECG amplitude data points having a slope that transgresses the slope threshold.

Clause 45: The method of any of clauses 41-44, wherein the second order short-term noise artifacts comprise at least one of a motion artifact, electrostatic interference, triboelectric interference, and electromagnetic interference.

Clause 46: The method of any of clauses 41-45, wherein the processor is further configured obtain at least two ECG signal channels from the plurality of physiological sensors.

Clause 47: The method of clause 46, wherein the at least two ECG signal channels comprise a primary ECG signal channel and a secondary ECG signal channel.

Clause 48: The method of clause 47, wherein the secondary ECG channel is provided to validate the presence of the second order short-term noise artifact.

Clause 49: The method of any of clauses 41-48, wherein the predetermined time windows have a duration of at least one of 1 ms, 10 ms, 100 ms, or 1000 ms.

Clause 50: The method of any of clauses 41-49, wherein the short-term noise artifact is a motion artifact that lasts for a duration of at least one of about 0.1 ms, 10 ms, 100 ms, 1 second, 2 seconds, 5 seconds, or 10 seconds.

Clause 51: The method of any of clauses 41-50, wherein the slope threshold is at least one of 2, 3, 5, or 7.

Clause 52: The method of any of clauses 41-51, wherein the slope threshold is determined based on a distribution of the determined slopes of the first order ECG data.

Clause 53: The method of any of clauses 41-52, wherein the representative ECG data point amplitudes are an average of amplitudes of the first order ECG data within the predetermined time windows.

Clause 54: The method of clause 53, wherein the average of the amplitudes of the first order ECG data is obtained using ensemble averaging.

Clause 55: A cardiac event monitoring system for removing short-term noise artifacts from an electrocardiogram (ECG) signal, comprising: a processor in communication with a wearable cardiac sensing device, the processor configured to receive ECG data based on electrical signal from a skin surface of a patient obtained from a plurality of physiological sensors of the wearable cardiac sensing device; obtain first order ECG data by processing the ECG data to remove first order artifacts; determine representative ECG data point amplitudes for predetermined time windows applied to the first order ECG data; determine a slope between substantially adjacent representative ECG amplitude data points; compare the slope to a slope threshold indicative of a presence of a second order short-term noise artifact; obtain second order ECG data by identifying segments of the first order ECG data corresponding to the representative ECG amplitude data points having a slope that transgresses the slope threshold; process the second order ECG data to compensate for the second order short-term noise artifacts to obtain processed ECG data; and determine whether an abnormal cardiac event has occurred based on the processed ECG data.

Clause 56: The cardiac event monitoring system of clause 55, wherein the second order ECG data is processed by removing the segments of the first order ECG data corresponding to the representative ECG amplitude data points having a slope that transgresses the slope threshold.

Clause 57: The cardiac event monitoring system of clause 55 or clause 56, wherein the second order ECG data is processed by averaging segments of the ECG data that neighbor the first order ECG data corresponding to the representative ECG amplitude data points having a slope that transgresses the slope threshold and replacing the first order ECG data corresponding to the representative ECG amplitude data points having a slope that transgresses the slope threshold with the average.

Clause 58: The cardiac event monitoring system of any of clauses 55-57, wherein the second order ECG data is processed by determining a noise score for the first order ECG data corresponding to the representative ECG amplitude data points having a slope that transgresses the slope threshold.

Clause 59: The cardiac event monitoring system of any of clauses 55-58, wherein the second order short-term noise artifacts comprise at least one of a motion artifact, electrostatic interference, triboelectric interference, and electromagnetic interference.

Clause 60: The cardiac event monitoring system of any of clauses 55-59, wherein the processor is further configured obtain at least two ECG signal channels from the plurality of physiological sensors.

Clause 61: The cardiac event monitoring system of clause 60, wherein the at least two ECG signal channels comprise a primary ECG signal channel and a secondary ECG signal channel.

Clause 62: The cardiac event monitoring system of clause 61, wherein the secondary ECG channel is provided to validate the presence of the second order short-term noise artifact.

Clause 63: The cardiac event monitoring system of any of clauses 55-62, wherein the predetermined time windows have a duration of at least one of 1 ms, 10 ms, 100 ms, or 1000 ms.

Clause 64: The cardiac event monitoring system of any of clauses 55-63, wherein the short-term noise artifact is a motion artifact that lasts for a duration of at least one of about 0.1 ms, 10 ms, 100 ms, 1 second, 2 seconds, 5 seconds, or 10 seconds.

Clause 65: The cardiac event monitoring system of any of clauses 55-64, wherein the slope threshold is at least one of 2, 3, 5, or 7.

Clause 66: The cardiac event monitoring system of any of clause 55-65, wherein the slope threshold is determined based on a distribution of the determined slopes of the first order ECG data.

Clause 67: The cardiac event monitoring system of any of clauses 55-66, wherein the representative ECG data point amplitudes are an average of amplitudes of the first order ECG data within the predetermined time windows.

Clause 68: The cardiac event monitoring system of clause 67, wherein the average of the amplitudes of the first order ECG data is obtained using ensemble averaging.

Clause 69: The cardiac event monitoring system of any of clauses 55-68, wherein the wearable cardiac sensing device further comprises a garment configured to be worn around the patient's torso for an extended period of time.

Clause 70: The cardiac event monitoring system of clause 69, wherein at least a portion of the plurality of physiological sensors are configured to be removably mounted onto the garment.

Clause 71: The cardiac event monitoring system of clause 69 or clause 70, wherein at least a portion of the plurality of physiological sensors are permanently integrated into the garment.

Clause 72: The cardiac event monitoring system of any of clauses 69-71, wherein the plurality of physiological sensors comprise at least three physiological sensors and the garment further includes one or more therapy electrodes incorporated therein, wherein the one or more therapy electrodes are positioned within the garment to deliver one or more therapeutic defibrillating shocks to the patient.

Clause 73: The cardiac event monitoring system of any of clauses 55-72, wherein the wearable cardiac sensing device further comprises a removable adhesive patch configured to be adhered to skin of the patient.

Clause 74: The cardiac event monitoring system of clause 73, wherein at least a portion of the plurality of physiological sensors are configured to be removably mounted onto the removable adhesive patch.

Clause 75: The cardiac event monitoring system of clause 73 or clause 74, wherein the wearable cardiac sensing device further comprises a cardiac sensing unit incorporating the portion of the plurality of physiological sensors, the cardiac sensing unit configured to be removably mounted onto the removable adhesive patch.

Clause 76: The cardiac event monitoring system of any of clauses 55-75, wherein the wearable cardiac sensing device further comprises at least one tissue fluid monitor configured to measure a fluid level in tissue of the patient.

Clause 77: The cardiac monitoring system of clause 76, wherein the at least one tissue fluid monitor comprises one or more antennas configured to direct RF waves through the tissue of the patient and measure output RF signals in response to the RF waves that have passed through the tissue.

Clause 78: The cardiac monitoring system of clause 76 or clause 77, wherein the output RF signals comprise parameters indicative of the fluid level in the tissue of the patient.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples, and are incorporated in and constitute a part of this specification, but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure.

FIG. 1 depicts an example wearable cardiac sensing system including a wearable cardiac sensing device.

FIG. 2 depicts an example garment-based monitoring device of a wearable cardiac sensing device.

FIG. 3 depicts an example electronic architecture for a garment-based monitoring device.

FIG. 4 depicts an example wearable cardiac sensing system including a wearable cardiac sensing device.

FIG. 5 depicts an example adhesive patch of a wearable cardiac sensing device.

FIG. 6 depicts an example cardiac sensing unit of a wearable cardiac sensing device.

FIG. 7 depicts an example of using an adhesive patch with a cardiac sensing unit.

FIG. 8 depicts an example electronic architecture for a cardiac sensing unit.

FIG. 9A depicts an example ECG signal obtained from a wearable cardiac sensing device.

FIG. 9B depicts an example ECG signal that has been processed to remove first order artifacts.

FIG. 10 depicts an example process flow for removing short-term noise artifacts from an electrocardiogram (ECG) signal.

FIG. 11 depicts first order ECG data.

FIG. 12 depicts first order ECG data having predetermined time windows applied thereto.

FIG. 13A depicts representative ECG amplitude data points.

FIG. 13B depicts a graph slope values between adjacent substantially adjacent representative ECG amplitude data points.

FIG. 14 depicts a graph slope values between adjacent substantially adjacent representative ECG amplitude data points having a slope threshold illustrated thereon.

FIG. 15 depicts a distribution of the determined slopes of the ECG data for determining the slope threshold.

FIG. 16A depicts an example screen that may be displayed to a patient.

FIG. 16B depicts another example screen that may be displayed to a patient.

FIG. 16C depicts another example screen that maybe displayed to a patient.

FIG. 17 depicts an example component view of a cardiac sensing unit.

FIG. 18 depicts another example of a wearable cardiac sensing device.

FIG. 19 depicts another example of a wearable cardiac sensing device.

FIG. 20 depicts another example of a wearable cardiac sensing device.

### DETAILED DESCRIPTION

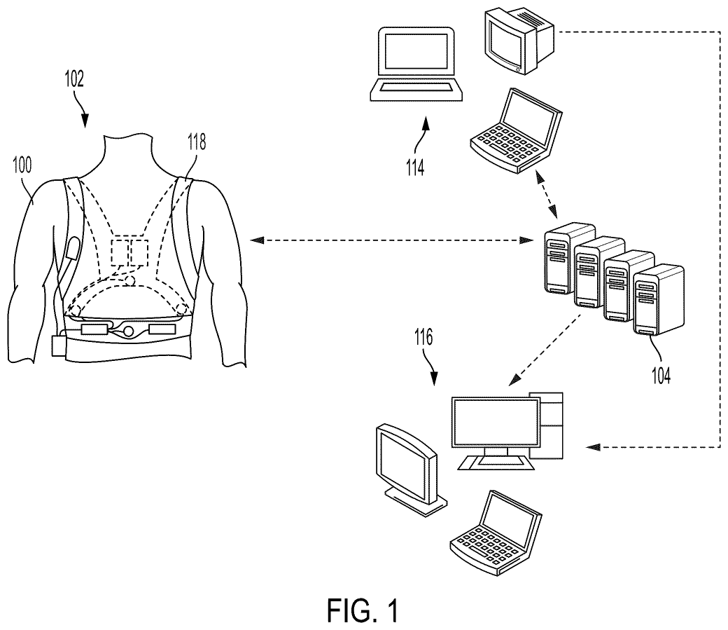

Cardiac event monitoring systems implementing the devices, methods, and techniques disclosed herein can be used to monitor patients with known or suspected cardiac conditions. For example, some patients with a suspected cardiac condition may be prescribed a wearable cardiac sensing device, or a wearable cardiac sensing device and treatment device, that the patient must wear for the prescribed period of time. During the period of wear, the cardiac sensing device generates ECG signals for the patient and may collect other data about the patient, such as data regarding the patient's movement, posture, lung fluid levels, respiration rate, and/or the like. The ECG signals are analyzed, at the cardiac sensing device and/or at a remote server in communication with the cardiac sensing device, to determine if the patient has a cardiac condition. Example cardiac conditions may include arrhythmias such as ventricular ectopic beats (VEB), ventricular runs/ventricular tachycardia, bigeminy, supraventricular ectopic beats (SVEB), supraventricular tachycardia, atrial fibrillation, ventricular fibrillation, pauses, 2^{nd} AV blocks, 3^{rd} AV blocks, bradycardia, and/or other types of tachycardia. As shown in FIG. 1, for example, a patient 100 with a known cardiac condition may be prescribed a wearable cardiac sensing and treatment device 102 configured to provide the patient 100 with a treatment, such as therapeutic defibrillation pulses, upon detecting certain types of arrhythmias that the device is configured to treat (e.g., ventricular fibrillation). The patient may similarly use the wearable cardiac sensing and treatment device for the prescribed period of time, while the device 102 and/or remote server 104 analyzes the patient's ECG signals to determine cardiac conditions in the patient.

As discussed in further detail below, the cardiac event monitoring systems implementing the devices, methods, and techniques disclosed herein may provide improved ECG signal analysis to determine abnormal cardiac conditions in a patient. The wearable cardiac sensing devices and the wearable cardiac sensing and treatment devices disclosed herein include ECG electrodes configured to sense electrical signals from a skin surface of the patient. ECG data is thereafter generated based on these electrical signals. In addition, these devices are configured to be worn by an ambulatory patient. Implementations herein include features for processing scenarios where when the ambulatory patient moves, noise in the form of motion artifacts might be introduced into the ECG data if the ECG electrodes move or shift on the patient's body. Implementations herein include features for processing scenarios where artifacts might also be introduced into the ECG data from other sources such as, but not limited to, electrostatic interference, electromagnetic interference, and/or triboelectric interference. Because ECG data is analyzed to determine abnormal cardiac conditions in a patient, implementations herein provide advantages of accurate determination of abnormal cardiac conditions even in scenarios where such artifacts are introduced into the ECG data. Implementations herein provide advantages of improved arrhythmia determination with a wearable cardiac sensing and/or treatment device in scenarios where a decision of the wearable cardiac treatment device to treat or not treat a patient with a therapeutic shock is based on the device's determination of an abnormal cardiac condition in the presence of short-term noise artifacts in the ECG data.

In various implementations herein, noise artifacts can be removed through first order noise artifact removal systems and/or processes, which are then followed by second order noise artifact removal systems and/or processes. In various implementations herein, first order noise artifact removal systems and/or processes can remove long term noise artifacts, e.g., those caused by repetitive interference patterns such as a recurring external electromagnetic interference signal with duration of longer than 10 seconds or more, e.g., 30 seconds or longer, 1 minute or longer, 1 hour or longer, and/or a continuously present interference effect in the underlying ECG data. In various implementations herein, long term noise artifacts can include baseline wander over a longer duration of the signal, e.g., with duration of longer than 10 seconds or more, e.g., 30 seconds or longer, 1 minute or longer, 1 hour or longer, and/or a continuously present wander effect in the underlying ECG data. In implementations described herein, short-term noise artifacts are removed after first order processing systems and/or techniques described above.

In example implementations herein, wearable cardiac treatment and/or detection systems and methods are configured to remove first order noise artifacts from ECG data before processing such ECG data for second order noise artifacts. For example, such first order artifact removal methods include filtering techniques for removal of baseline wander, signal spikes, and/or interference from other neighboring devices and/or systems. For example, first order noise artifact removal can be based on a finite impulse response (FIR) that may be utilized to remove noise or artifacts from the ECG data. In addition or alternatively, a frequency-based Fast Fourier Transform (FFT) detector that applies a full spectrum filtering may be utilized. In other implementations herein, a low-pass filter may be utilized to eliminate high frequency components from the ECG data, a median filter may be applied for baseline removal by centering the ECG data at zero amplitude and flattening the wandering baseline and to eliminate low frequency components, and a notch filter may be provided to eliminate AC interference. The use of such filtering techniques to process the ECG data to remove first order artifacts thus can provide first order ECG data that is available for subsequent processing.

Such first order ECG data may, in some cases, still include artifacts caused by short-term events because such filtering techniques are not sensitive to the presence of short-term noise artifacts. Typically, these short term events include motion by the patient causing a brief shifting of the ECG electrodes but may also include short term electrostatic interference, electromagnetic interference, and/or triboelectric interference. In examples, short term events may have a duration of at least one of about 0.1 ms, 10 ms, 100 ms, 1 second, 2 seconds, 5 seconds, or 10 seconds. Accordingly, the devices, methods, and techniques disclosed herein provide the ability to compensate the first order ECG data for these short term noise artifacts and provide second order data. The analysis of such second order ECG data provides improved ECG signal analysis to determine abnormal cardiac conditions in a patient when compared to conventional ECG processing systems, techniques and/or methods to determine abnormal cardiac conditions.

These and other features and characteristics of the present disclosure, as well as the methods of operation and functions of the related elements of structures and the combination of parts and economics of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limit of the disclosure.

As used herein, the singular form of “a”, “an”, and “the” include plural referents unless the context clearly dictates otherwise.

As used herein, the terms “right”, “left”, “top”, and derivatives thereof shall relate to aspects of the present disclosure as it is oriented in the drawing figures. However, it is to be understood that embodiments of the present disclosure can assume various alternative orientations and, accordingly, such terms are not to be considered as limiting. Also, it is to be understood that embodiments of the present disclosure can assume various alternative variations and stage sequences, except where expressly specified to the contrary. It is also to be understood that the specific devices and processes illustrated in the attached drawings, and described in the following specification, are provided as examples. Hence, specific dimensions and other physical characteristics related to the embodiments disclosed herein are not to be considered as limiting.

As used herein, including in the claims, unless otherwise stated, a statement that a function or operation is “based on” an item or condition means that the function or operation is based on the stated item or condition and may be based on one or more items and/or conditions in addition to the stated item or condition.

As used herein, the terms “communication” and “communicate” refer to the receipt or transfer of one or more signals, messages, commands, or other type of data. For one unit or component to be in communication with another unit or component means that the one unit or component is able to directly or indirectly receive data from and/or transmit data to the other unit or component. This can refer to a direct or indirect connection that can be wired and/or wireless in nature. Additionally, two units or components can be in communication with each other even though the data transmitted can be modified, processed, routed, and the like, between the first and second unit or component. For example, a first unit can be in communication with a second unit even though the first unit passively receives data, and does not actively transmit data to the second unit. As another example, a first unit can be in communication with a second unit if an intermediary unit processes data from one unit and transmits processed data to the second unit. It will be appreciated that numerous other arrangements are possible.

For convenience, the present disclosure presents devices, methods, and techniques for removing short-term noise artifacts from ECG data that are broadly presented as follows.

In one implementation, a cardiac event monitoring system for removing short-term noise artifacts from an electrocardiogram (ECG) signal is provided. The system includes a wearable cardiac sensing device configured to be bodily-attached to the patient. The wearable cardiac sensing device comprises a plurality of physiological sensors comprising one or more ECG electrodes configured to sense electrical signals from a skin surface of the patient; and a non-transitory memory configured to store ECG data based on the electrical signals of the patient. The system also includes a processor in communication with the wearable cardiac sensing device. The processor is configured to receive the ECG data from the memory; obtain first order ECG data by processing the ECG data to remove first order artifacts; determine representative ECG data point amplitudes for predetermined time windows applied to the first order ECG data; determine a slope between substantially adjacent representative ECG amplitude data points; compare the slope to a slope threshold indicative of a presence of a second order short-term noise artifact; obtain second order ECG data by identifying segments of the first order ECG data corresponding to the representative ECG amplitude data points having a slope that transgresses the slope threshold; process the second order ECG data to compensate for the second order short-term noise artifacts to obtain processed ECG data; and determine whether an abnormal cardiac event has occurred based on the processed ECG data.

In another implementation, a non-transitory computer-readable medium storing sequences of instructions executable by at least one processor is provided. The sequences of instructions instruct the at least one processor to: receive ECG data; obtain first order ECG data by processing the ECG data to remove first order artifacts; determine representative ECG data point amplitudes for predetermined time windows applied to the first order ECG data; determine a slope between substantially adjacent representative ECG amplitude data points; compare the slope to a slope threshold indicative of a presence of a second order short-term noise artifact; obtain second order ECG data by identifying segments of the first order ECG data corresponding to the representative ECG amplitude data points having a slope that transgresses the slope threshold; process the second order ECG data to compensate for the second order short-term noise artifacts to obtain processed ECG data; and determine whether an abnormal cardiac event has occurred based on the processed ECG data.

In one implementation, a method for removing short-term noise artifacts from an electrocardiogram (ECG) signal is provided. The method comprises: receiving ECG data; obtaining first order ECG data by processing the ECG data to remove first order artifacts; determining representative ECG data point amplitudes for predetermined time windows applied to the first order ECG data; determining a slope between substantially adjacent representative ECG amplitude data points; comparing the slope to a slope threshold indicative of a presence of a second order short-term noise artifact; obtaining second order ECG data by identifying segments of the first order ECG data corresponding to the representative ECG amplitude data points having a slope that transgresses the slope threshold; processing the second order ECG data to compensate for the second order short-term noise artifacts to obtain processed ECG data; and determining whether an abnormal cardiac event has occurred based on the processed ECG data.

In another implementation, a cardiac event monitoring system for removing short-term noise artifacts from an electrocardiogram (ECG) signal is provided. The system comprises a processor in communication with a wearable cardiac sensing device. The processor is configured to receive ECG data based on electrical signal from a skin surface of a patient obtained from a plurality of physiological sensors of the wearable cardiac sensing device; obtain first order ECG data by processing the ECG data to remove first order artifacts; determine representative ECG data point amplitudes for predetermined time windows applied to the first order ECG data; determine a slope between substantially adjacent representative ECG amplitude data points; compare the slope to a slope threshold indicative of a presence of a second order short-term noise artifact; obtain second order ECG data by identifying segments of the first order ECG data corresponding to the representative ECG amplitude data points having a slope that transgresses the slope threshold; process the second order ECG data to compensate for the second order short-term noise artifacts to obtain processed ECG data; and determine whether an abnormal cardiac event has occurred based on the processed ECG data.

In one example use case, a cardiologist may prescribe a patient with a known cardiac condition a wearable cardiac sensing device with treatment capabilities. For example, the wearable cardiac sensing device may be configured to provide cardioversion, defibrillation, and/or pacing (e.g., anti-bradycardic or anti-tachycardic) treatment pulses to the patient upon detecting an arrhythmia in the patient that the device is configured to treat. For example, the wearable cardiac sensing device may be a wearable cardioverter defibrillator (WCD). The wearable cardiac sensing and treatment device may be in direct communication with a remote server (e.g., be configured as a garment-based monitoring and treatment device in direct communication with the remote server). In implementations, the wearable cardiac sensing device may be configured as a garment-based sensing device including a garment where monitoring and treatment components of the garment-based sensing device are configured to be removably mounted onto the garment. Alternatively, at least some of the monitoring and treatment components of the garment-based sensing device may be permanently disposed on the garment. For example, the garment may include ECG electrodes and/or treatment electrodes sewn into, adhered onto, incorporated into the threads of, or otherwise permanently included as part of the garment. The patient wears the garment-based sensing device under his/her clothes for the prescription period, which may be the period between when the patient is diagnosed with the known cardiac condition and when the patient is scheduled to receive an implantable treatment device for the cardiac condition, such as an implantable defibrillator. In other situations, the patient may wear the garment-based sensing device for a period of time as the patient undergoes cardiac rehabilitation to improve their cardiac condition to the point that they no longer need to be protected by a treatment device. Alternatively, in some implementations, the wearable cardiac sensing device may be configured as an adhesive patch removably attached to the body of the patient and configured to support both monitoring and treatment components disposed on the patch. Such a wearable cardiac sensing device with treatment capabilities may be in direct communication with a remote server. In some examples, such a wearable cardiac sensing device with treatment capabilities may be in direct communication with a portable gateway device, which in term may be in communication with a remote server. At the wearable cardiac sensing device, the portable gateway, and/or at the remote server, the ECG signals may be subjected to processing to remove short-term noise artifacts therefrom as described in greater detail hereinafter. Based on the processed ECG signals with the short-term noise artifacts removed, the wearable cardiac sensing device, the portable gateway and/or the remote server may determine if the patient is expressing abnormal cardiac events prior to issuing instructions to a processor of the wearable cardiac sensing device to issue one or more therapeutic pulses to the patient in accordance with the principles described herein. For example, a WCD can use the processed ECG signals with the short-term noise artifacts removed based on the techniques described herein to determine whether or not to issue cardioversion, defibrillation, and/or pacing (e.g., anti-bradycardic or anti-tachycardic) treatment pulses to the patient upon detecting an arrhythmia in the patient that the WCD is configured to treat.

In another example use case, a cardiologist may prescribe that a patient with a suspected cardiac condition use a wearable cardiac sensing device for a prescribed time period. The wearable cardiac sensing device may include an adhesive patch, a cardiac sensing unit configured to be mounted onto the adhesive patch, and a portable gateway in communication with the cardiac sensing unit and with a remote server. The patient may use the wearable cardiac sensing device during his/her daily activities (e.g., with the adhesive patch and cardiac sensing unit worn under the patient's clothes and the portable gateway carried with the patient) during the prescribed time period. As the wearable cardiac sensing device is being used by the patient, the wearable cardiac sensing device provides ECG signals for the patient based on sensed electrical activity. At the wearable cardiac sensing device, the portable gateway, and/or at the remote server, the ECG signals may be subjected to processing to remove short-term noise artifacts therefrom as described in greater detail hereinafter. Based on the processed ECG signals with the short-term noise artifacts removed, the wearable cardiac sensing device, the portable gateway and/or the remote server may determine if the patient is expressing abnormal cardiac events for recording, reporting, and/or notification purposes.

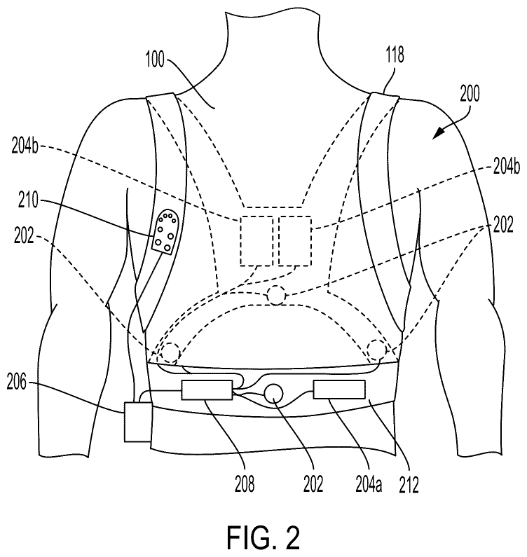

FIG. 1 illustrates an example of a cardiac event monitoring system for removing first order artifacts followed by removal of second order short-term noise artifacts from an electrocardiogram (ECG) signal gathered from a patient 100, according to implementations disclosed herein. The cardiac event monitoring system of FIG. 1 includes a wearable cardiac sensing device 102 configured to be worn by the patient 100, where the wearable cardiac sensing device 102 is in communication with a remote server 104. The wearable cardiac sensing device 102 according to FIG. 1 is a garment-based sensing device 118 configured to be worn about the patient's torso for an extended period of time, where various electronic components are temporarily and/or permanently mounted to the garment of the garment-based sensing device 118 (as described in further detail with respect to FIG. 2 below). However, other implementations of the wearable cardiac sensing system may include different embodiments of a wearable cardiac sensing device 102, as described in further detail below.

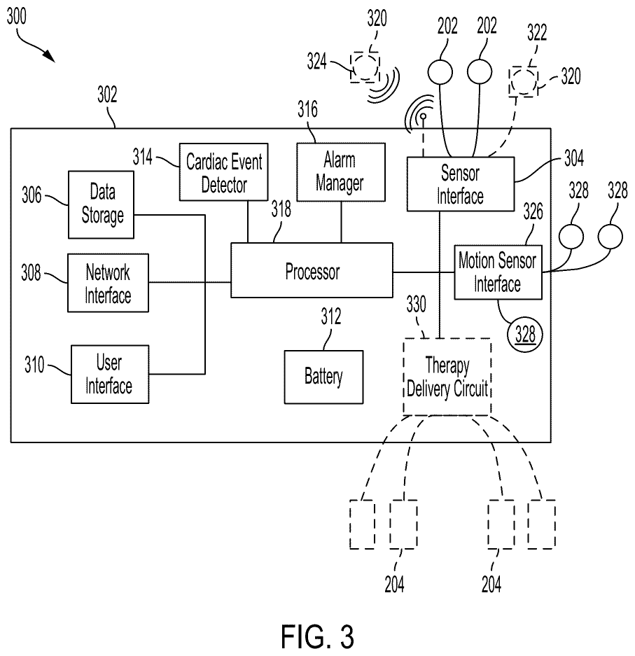

The garment-based sensing device 118 of FIG. 1 can be, for example, capable of and designed for moving with the patient 100 as the patient 100 goes about his/her daily routine. For example, the garment-based sensing device 118 as described herein with respect to FIG. 1 (as well as FIGS. 2 and 3 below) can be a wearable cardioverter defibrillator, or wearable defibrillator, configured to be bodily-attached to the patient 100. In one example scenario, such wearable defibrillators can be worn nearly continuously or substantially continuously for a week, two weeks, a month, or two or three months at a time. During the period of wear, the patient can be encouraged to engage in normal activities at home and at work. For example, the patient may work in the garden or cook and/or clean a room while wearing the device. During such activities, the patient may move their body, including the upper torso area about where the device and/or portions of the device (e.g., garment, adhesive electrodes, therapy electrodes and so on) are attached or worn. Implementations herein allow for robust ECG data collection during such movements. During the period of time in which it is worn by the patient 100, the wearable defibrillator can be configured to continuously or substantially continuously monitor the vital signs of the patient 100 and, upon determining that the patient 100 is experiencing an arrhythmia that the wearable defibrillator is configured to treat based on the processed ECG signals with the short-term noise artifacts removed, deliver one or more therapeutic electrical pulses to the patient 100. For example, such therapeutic shocks can be pacing, defibrillation, cardioversion, or transcutaneous electrical nerve stimulation (TENS) pulses.

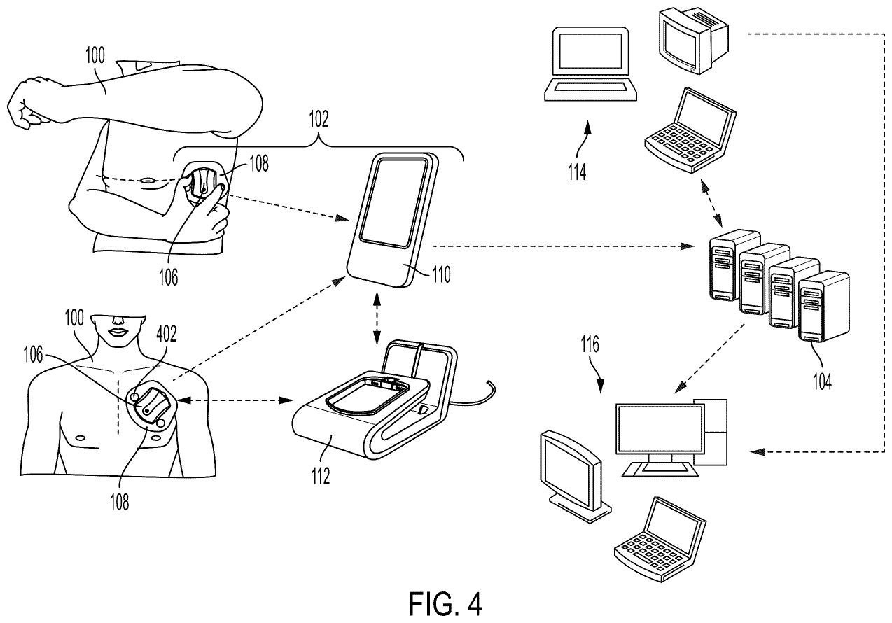

The wearable cardiac sensing device 102 may be configured to transmit ECG data and/or ECG signals directly to the remote server 104. Accordingly, the wearable cardiac sensing device 102 may be in wired or wireless communication with the remote server 104. As an illustration, the wearable cardiac sensing device 102 may communicate with the remote server 104 via Ethernet, via Wi-Fi, via near-field communication (NFC), via radiofrequency, via cellular networks, via Bluetooth®-to-TCP/IP access point communication, and/or the like. Accordingly, the wearable cardiac sensing device 102 may include communications circuitry configured to implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In implementations, as indicated above, the communications circuitry in the wearable cardiac sensing device 102 may be part of an IoT and communicate with the remote server 104 via IoT protocols for handling secure (e.g., encrypted) messaging and routing. Alternatively or additionally, the example wearable cardiac sensing device 102 may also include a portable gateway, similar to the portable gateway 110 shown and described below with reference to FIG. 4, where the portable gateway facilitates communication between the garment-based sensing device 118 and the remote server 104. The portable gateway may, additionally or alternatively, be configured to perform some or all of the analysis described below.

The remote server 104 is configured to receive and process the ECG data and/or ECG signals received from the wearable cardiac sensing device 102. In implementations, the remote server 104 may be in electronic communication with a number of wearable cardiac sensing devices 102 and be configured to receive and process the ECG data and/or ECG signals received from all of the wearable cardiac sensing devices 102 in communication with the remote server 104. The remote server 104 may include a computing device, or a network of computing devices, including at least one database (e.g., implemented in non-transitory media or memory) and at least one processor configured to execute sequences of instructions (e.g., stored in the database, with the at least one processor being in communication with the database) to receive and process the data and/or signals received from the wearable cardiac sensing device 102. For example, the at least one processor of the remote server 104 may be implemented as a digital signal processor (DSP), such as a 24-bit DSP processor; as a multicore-processor (e.g., having two or more processing cores); as an Advanced RISC Machine (ARM) processor, such as a 32-bit ARM processor; and/or the like. The at least one processor of the remote server 104 can execute an embedded operating system and further execute services provided by the operating system, where these services can be used for file system manipulation, display and audio generation, basic networking, firewalling, data encryption, communications, and/or the like. The database may be implemented as flash memory, solid state memory, magnetic memory, optical memory, cache memory, combinations thereof, and/or others. In various implementations, the remote server 104 may use the data received from the wearable cardiac sensing device 102 to perform first order processing, and further, remove short-term noise artifacts during second order processing from the ECG signal. The device 102 can then determine abnormal cardiac events as described in further detail below. Alternatively, in some implementations, the wearable cardiac sensing device 102 may perform some or all of the analysis described herein as being performed by the remote server 104 (e.g., with the wearable cardiac sensing device 102 transmitting an indication of an abnormal cardiac event in the patient 100 or another output to the remote server 104).

As shown in FIG. 1, in implementations, the cardiac event monitoring system further includes one or more user interfaces, such as technician interfaces 114 and caregiver interfaces 116. The technician interfaces 114 and caregiver interfaces 116 are in electronic communication with the remote server 104 through a wired or wireless connection. For instance, the technician interfaces 114 and caregiver interfaces 116 may communicate with the remote server 104 via Wi-Fi, via Ethernet, via cellular networks, and/or the like. Additionally, as shown, at least some of the technician interfaces 114 may be in electronic communication with at least some of the caregiver interfaces 116 through a wired or wireless connection, such as via Wi-Fi, via Ethernet, via cellular networks, and/or the like. The technician interfaces 114 and the caregiver interfaces 116 may include, for example, desktop computers, laptop computers, and/or portable personal digital assistants (e.g., smartphones, tablet computers, etc.).

In implementations, the technician interfaces 114 are configured to electronically communicate with the remote server 104 for the purpose of viewing and analyzing information gathered from one or more wearable cardiac sensing devices 102. For example, a technician interface 114 may provide one or more instructions to the remote server 104 to prepare a report on data and/or signals received from a given wearable cardiac sensing device 102 for a certain time period. Accordingly, a technician interface 114 may include a computing device having a processor communicably connected to a memory and a visual display. The technician interface 114 may display to a user of the technician interface 114 (e.g., a technician) data received from a wearable cardiac sensing device 102 and/or information computed from the data and/or signals received from wearable cardiac sensing device 102, described in further detail below. The user of the technician interface 114 may then provide one or more inputs to the remote server 104 to guide the remote server 104 in, for example, preparing a report for a patient 100.

As an illustration, a user of a technician interface 114 may select a time period to use for a report, and the remote server 104 may prepare a report corresponding to the selected time period. As another example, a user of a technician interface 114 may select types of data to be included in a report, such as certain abnormal cardiac events as described in more detail below. The remote server 104 may then prepare a report according to the types of data selected by the user. As another example, a user of a technician interface 114 may view a report prepared by the remote server 104 and draft a summary of the report to be included in a summary section for the report. Alternatively, in implementations, the remote server 104 may analyze, summarize, etc. the ECG data and/or ECG signals received from a wearable cardiac sensing device 102 with minimal or no input or interaction with a technician interface 114. In this way, the remote server 104 may analyze, summarize, etc. the information gathered from a wearable cardiac sensing device 102 and prepare a report on this information through a completely or mostly automated process. Such reports provide the user with clinically actionable information based on the processed ECG signals with the short-term noise artifacts removed.

The caregiver interfaces 116 are configured to electronically communicate with the remote server 104 for the purpose of viewing information on various patients 100 using a wearable cardiac sensing device 102. As such, a caregiver interface 116 may include a computing device having a processor communicably connected to a memory and a visual display. The caregiver interface 116 may display to a user of the caregiver interface 116 (e.g., a physician, a nurse, or other caregiver), for example, abnormal cardiac events detected for the patient 100 using the wearable cardiac sensing device 102, as described in further detail below. In implementations, the caregiver interface 116 may display to a user one or more reports summarizing abnormal cardiac events in the patient 100, such as one or more reports prepared by the remote server 104 (e.g., based on inputs from one or more technician interfaces 114). In implementations, the user of a caregiver interface 116 may be able to interact with the information displayed on the caregiver interface 116. As an example, the user of a caregiver interface 116 may be able to select a portion of a patient report and, in response, be able to view additional information relating to the selected portion of the report. Additional information may include, for instance, ECG data from the wearable cardiac sensing device 102 used to prepare the report and/or the like. In implementations, the user of the caregiver interface 116 may instead view a static patient report that does not have interactive features.

In implementations, a technician interface 114 and/or a caregiver interface 116 may be a specialized interface configured to communicate with the remote server 104. As an example, the technician interface 114 may be a specialized computing device configured to receive preliminary patient reports from the remote server 104, receive inputs from a user to adjust the preliminary report, and transmit the inputs back to the remote server 104. The remote server 104 then uses the inputs from the technician interface 114 to prepare a finalized patient report, which the remote server 104 also transits to the technician interface 114 for review by the user. As another example, the caregiver interface 116 may be a specialized computing device configured to communicate with the remote server 104 to receive and display patient reports, as well as other information regarding patients 100 using a wearable cardiac sensing device 102.

In implementations, a technician interface 114 and/or a caregiver interface 116 may be a generalized user interface that has been adapted to communicate with the remote server 104. To illustrate, the technician interface 114 may be a computing device (e.g., a laptop, a portable personal digital assistant such as a smartphone or tablet, etc.) executing a technician application that configures the computing device to communicate with the remote server 104. For example, the technician application may be downloaded from an application store or otherwise installed on the computing device. Accordingly, when the computing device executes the technician application, the computing device is configured to establish an electronic communication link with the remote server 104 to receive and transmit information regarding patients 100 using a wearable cardiac sensing device 102. Similarly, the caregiver interface 116 may be a computing device (e.g., a laptop, a portable personal digital assistant such as a smartphone or tablet, etc.) executing a caregiver application that configures the computing device to communicate with the remote server 104. The caregiver application may be similarly downloaded from an application store or otherwise installed on the computing device and, when executed, may configure the computing device to establish a communication link with the remote server 104 to receive and display information on patients 100 using a wearable cardiac sensing device 102.

The application store is typically included within an operating system of a computing device implementing a user interface. For example, in a device implementing an operating system provided by Apple Inc. (Cupertino, California), the application store can be the App Store, a digital distribution platform, developed and maintained by Apple Inc., for mobile apps on its iOS and iPadOS® operating systems. The application store allows a user to browse and download an application, such as the technician or caregiver application, developed in accordance with the Apple® iOS Software Development Kit. For instance, such technician or caregiver application may be downloaded on an iPhone® smartphone, an iPod Touch® handheld computer, or an iPad® tablet computer, or transferred to an Apple Watch® smartwatch. Other application stores may alternatively be used for other types of computing devices, such as computing devices operating on the Android® operating system.

In some implementations, the technician application and the caregiver application may be the same application, and the application may provide different functionalities to the computing device executing the application based on, for example, credentials provided by the user. For instance, the application may provide technician functionalities to a first computing device in response to authenticating technician credentials entered on the first computing device, and may provide caregiver functionalities to a second computing device in response to authenticating caregiver credentials entered on the second computing device. In other cases, the technician application and the caregiver application may be separate applications, each providing separate functionalities to a computing device executing them.

In implementations, the cardiac event monitoring system shown in FIG. 1 may include other types of interfaces. To illustrate, in examples, the cardiac event monitoring system may include patient interfaces. The remote server 104 and/or a technician interface 114 may provide a report on a patient 100 using a wearable cardiac sensing device 102 to the patient 100 via a patient interface. This patient report may be the same as a report provided to a caregiver via a caregiver interface 116, or this report may be different from the report provided to the caregiver via the caregiver interface 116. For instance, the report provided to a patient 100 may be an abridged version of a report prepared for the patient's caregiver, such as a report that summarizes the key information from the report prepared for the patient's caregiver. In various implementations, the patient interface may be configured similarly to and function similarly to the caregiver interface 116 discussed above. As an example, the patient interface may be similar to the caregiver interface 116 but with some restrictions on what is included in a patient report compared to a report prepared for the patient's caregiver, and/or with some restrictions on the functionalities of, for instance, an application executed by the patient interface that the patient 100 can use to access and view a patient report or other information on the patient 100.

With reference to FIG. 2 and with continuing reference to FIG. 1, the garment-based sensing device 118 is illustrated in more detail, according to various implementations. As shown in FIG. 2, the garment-based sensing device 118 can include one or more of the following: a garment 200 configured to be worn around the patient's torso, a plurality of physiological sensors, one or more therapy electrodes 204a and 204b (collectively referred to herein as therapy electrodes 204), a medical device controller 206, a connection pod 208, a patient interface pod 210, a belt 212, or any combination of these components. In examples, at least some of the components of the garment-based sensing device 118 (e.g., at least a portion of the physiological sensors, including the ECG electrodes 202; at least a portion of the therapy electrodes 204; etc.) are configured to be mounted on or affixed to the garment 200, such as by mating hooks, hook-and-loop fabric strips, receptacles (e.g., pockets), and the like. For example, the ECG electrodes 202 may be mounted on the garment 200 by hook-and-loop fabric strips on the electrodes 202 and the garment 200, and the therapy electrodes 204 may be mounted on the garment 200 by being inserted into receptacles of the garment 200 (e.g., into pockets of the garment 200). In examples, at least some of the components of the garment-based sensing device 118 (e.g., at least a portion of the physiological sensors, including the ECG electrodes 202; at least a portion of the therapy electrodes 204; etc.) can be permanently integrated into the garment 200, such as being sewn into or permanently adhered onto the garment 200. In examples, at least some of the components may be connected to each other through external cables, through sewn-in connections (e.g., wires woven into the fabric of the garment 200 or between layers of the garment 200), through conductive fabric of the garment 200, and/or the like.

The medical device controller 206 can be operatively coupled to the ECG electrodes 202, which can be affixed to the garment 200 (e.g., assembled into the garment 200 or removably attached to the garment 200, for example, using hook-and-loop fasteners) or permanently integrated into the garment 200. In implementations, the medical device controller 206 is also operatively coupled to the therapy electrodes 204. The therapy electrodes 204 may be similarly assembled into the garment 200 (e.g., into pockets or other receptacles of the garment 200) or permanently integrated into the garment 200. As shown in FIG. 2, the ECG electrodes 202 and/or the therapy electrodes 204 can be directly operatively coupled to the medical device controller 206 through the connection pod 208. Component configurations other than those shown in FIG. 2 are also possible. For example, the ECG electrodes 202 can be configured to be attached at various positions about the body of the patient 100. In certain implementations, signals from various combinations of pairs of the plurality of ECG electrodes 202 can be used to define different channels of the ECG signal. For example, signals measured using a particular pair of electrodes can be used to obtain a differential signal to define a corresponding channel of the ECG signal. In some implementations, a first ECG channel may be associated with a first electrode positioned proximate a front of the patient 100 and a second electrode positioned proximate a back of the patient (e.g., a front-to-back (FB) channel). Additionally or alternatively, a second ECG channel may be associated with a third electrode positioned proximate a first side of the patient and a fourth electrode positioned proximate a second side of the patient (e.g., a side-to-side (SS) channel). Additionally or alternatively, the FB channel may be orthogonal to the SS channel (e.g., a first hypothetical line connecting the first electrode to the second electrode may be substantially (e.g., approximately and/or the like) orthogonal (e.g., perpendicular and/or the like) to a second hypothetical line connecting the third electrode to the fourth electrode, the second hypothetical line may have a component (e.g., vector component, vector projection, and/or the like that may be calculated and/or the like) that is orthogonal to the first hypothetical line, and/or the like).

In implementations, at least one of the ECG electrodes 202 and/or at least one of the therapy electrodes 204 can be included on a single integrated patch and adhesively applied to the patient's body. In implementations, at least one of the ECG electrodes 202 and/or at least one of the therapy electrodes 204 can be included in multiple patches and adhesively applied to the patient's body. Such patches may be in wired (e.g., directly or via the connection pod 208) or wireless connection with the medical device controller 206. Similar implementations may also be extended to non-ECG physiological sensors of the garment-based sensing device 118. Such non-ECG physiological sensors may include, for instance, a motion sensor such as an accelerometer, a respiration sensor, a bioacoustics sensor, a blood pressure sensor, a temperature sensor, a pressure sensor, a humidity sensor, a P-wave sensor (e.g., a sensor configured to monitor and isolate P-waves within an ECG waveform), an oxygen saturation sensor (e.g., implemented through photoplethysmography, such as through light sources and light sensors configured to transmit light into the patient's body and receive transmitted and/or reflected light containing information about the patient's oxygen saturation), a fluid sensor configured to measure a fluid level in tissue of the patient using RF waves, and so on.

The ECG electrodes 202 are configured to detect one or more cardiac signals, such as electrical signals indicative of ECG activity from a skin surface of the patient 100. In implementations, the therapy electrodes 204 can also be configured to include sensors that detect ECG signals as well as, or in the alternative from, other physiological signals from the patient 100. The connection pod 208 can, in various examples, include a signal processor configured to amplify, filter, and digitize these cardiac signals prior to transmitting the cardiac signals to the medical device controller 206. In certain implementations, the filtering of the ECG signals performed by the signal processor of the connection pod 208 can obtain the first order ECG data with the first order artifacts removed.

Additionally, the therapy electrodes 204 can be configured to deliver one or more therapeutic cardioversion/defibrillation shocks to the body of the patient 100 when the medical device controller 206 determines that such treatment is warranted based on the signals detected by the ECG electrodes 202 and processed by the medical device controller 206. Example therapy electrodes 204 can include conductive metal electrodes such as stainless-steel electrodes. In implementations, the therapy electrodes 204 may also include one or more conductive gel deployment devices configured to deliver conductive gel between the metal electrode and the patient's skin prior to delivery of a therapeutic shock.

In implementations, the medical device controller 206 may also be configured to warn the patient 100 prior to the delivery of a therapeutic shock, such as via output devices integrated into or connected to the medical device controller 206, the connection pod 208, and/or the patient interface pod 210. The warning may be auditory (e.g., a siren alarm, a voice instruction indicating that the patient 100 is going to be shocked), visual (e.g., flashing lights on the medical device controller 206), haptic (e.g., a tactile, buzzing alarm generated by the connection pod 208), and/or the like. If the patient 100 is still conscious, the patient 100 may be able to delay or stop the delivery of the therapeutic shock. For example, the patient 100 may press one or more buttons on the patient interface pod 210 and/or the medical device controller 206 to indicate that the patient 100 is still conscious. In response to the patient 100 pushing the one or more buttons, the medical device controller 206 may delay or stop the delivery of the therapeutic shock.

In implementations, a garment-based sensing device 118 as described herein can be configured to switch between a therapeutic mode and a monitoring mode such that, when in the monitoring mode, the garment-based sensing device 118 is configured to only monitor the patient 100 (e.g., not provide or perform any therapeutic functions). For example, in such implementations, therapeutic components such as the therapy electrodes 204 and associated circuitry may be decoupled from (or coupled to) or switch out of (or switched into) the garment-based sensing device 118. As an illustration, a garment-based sensing device 118 can have optional therapeutic elements (e.g., defibrillation and/or pacing electrode components and associated circuitry) that are configured to operate in a therapeutic mode. The optional therapeutic elements may be physically decoupled from the garment-based sensing device 118 as a means to convert the garment-based sensing device 118 from a therapeutic mode into a monitoring mode. Alternatively, the optional therapeutic elements may be deactivated (e.g., by means of a physical or software switch), essentially rendering the garment-based sensing device 118 as a monitoring-only device for a specific physiological purpose for the particular patient 100. As an example of a software switch, an authorized person may be able to access a protected user interface of the garment-based sensing device 118 and select a preconfigured option or perform some other user action via the user interface to deactivate the therapeutic elements of the garment-based sensing device 118.

FIG. 3 illustrates a sample component-level view of a medical device controller 300 included in a garment-based sensing device 118. The medical device controller 300 is an example of the medical device controller 206 shown in FIG. 2 and described above. As shown in FIG. 3, the medical device controller 300 may include a housing 302 configured to house a number of electronic components, including a sensor interface 304, a data storage 306, a network interface 308, a user interface 310, at least one battery 312 (e.g., positioned within a battery chamber configured for such a purpose), a cardiac event detector 314, an alarm manager 316, at least one processor 318, and a therapy delivery circuit 330. As described above, in some implementations, the garment-based sensing device 118 may include like components to those described above but may not include therapeutic components. That is, in some implementations, the garment-based sensing device 118 can include ECG monitoring components and not provide therapy to the patient 100. Accordingly, the garment-based sensing device 118 may not include the therapy delivery circuit 330 and the therapy electrodes 204 (shown in dotted lines), or the therapy delivery circuit 330 and the therapy electrodes 204 may be disconnectable from the rest of the garment-based sensing device 118.

In implementations, the processor 318 includes one or more processors (or one or more processor cores) that are each configured to perform a series of instructions that result in the manipulation of data and/or the control of the operation of the other components of the medical device controller 300. In implementations, when executing a specific process (e.g., monitoring sensed electrical data of the patient 100), the processor 318 can be configured to make specific logic-based determinations based on input data received. The processor 318 may be further configured to provide one or more outputs that can be used to control or otherwise inform subsequent processing to be carried out by the processor 318 and/or other processors or circuitry to which the processor 318 is communicably coupled. Thus, the processor 318 reacts to a specific input stimulus in a specific way and generates a corresponding output based on that input stimulus. In example cases, the processor 318 can proceed through a sequence of logical transitions in which various internal register states and/or other bit cell states internal or external to the processor 318 may be set to logic high or logic low.

As referred to herein, the processor 318 can be configured to execute a function where software is stored in a data store (e.g., the data storage 306) coupled to the processor 318, the software being configured to cause the processor 318 to proceed through a sequence of various logic decisions that result in the function being executed. The various components that are described herein as being executable by the processor 318 can be implemented in various forms of specialized hardware, software, or a combination thereof. For example, the processor 318 can be a digital signal processor (DSP) such as a 24-bit DSP processor. As another example, the processor 318 can be a multi-core processor, e.g., having two or more processing cores. As another example, the processor 518 can be an Advanced RISC Machine (ARM) processor, such as a 32-bit ARM processor. The processor 318 can execute an embedded operating system and further execute services provided by the operating system, where these services can be used for file system manipulation, display and audio generation, basic networking, firewalling, data encryption, communications, and/or the like.

The data storage 306 can include one or more of non-transitory media, such as flash memory, solid state memory, magnetic memory, optical memory, cache memory, combinations thereof, and others. The data storage 306 can be configured to store executable instructions and data used for operation of the medical device controller 300. In implementations, the data storage 306 can include sequences of executable instructions that, when executed, are configured to cause the processor 318 to perform one or more functions. Additionally, the data storage 306 can be configured to store information such as digitized ECG signals of the patient 100.

In examples, the network interface 308 can facilitate the communication of information between the medical device controller 300 and one or more devices or entities over a communications network. For example, the network interface 308 can be configured to communicate with the remote server 104 or other similar computing device. Using the network interface 308, the garment-based sensing device 118 may transmit, for example, ECG signals, other physiological signals, indications of abnormal cardiac events, etc., to the remote server 104. In implementations, the network interface 308 can include communications circuitry for transmitting data in accordance with a Bluetooth® wireless standard for exchanging such data over short distances to an intermediary device(s) (e.g., a base station, “hotspot” device, smartphone, tablet, portable computing device, and/or other device in proximity with the garment-based sensing device 118). The intermediary device(s) may in turn communicate the data to the remote server 104 over a broadband cellular network communications link. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) may communicate with the remote server 104 over a Wi-Fi communications link based on the IEEE 802.11 standard. In implementations, the network interface 308 may be configured to instead communicate directly with the remote server 104 without the use of intermediary device(s). In such implementations, the network interface 308 may use any of the communications links and/or protocols provided above to communicate directly with the remote server 104.

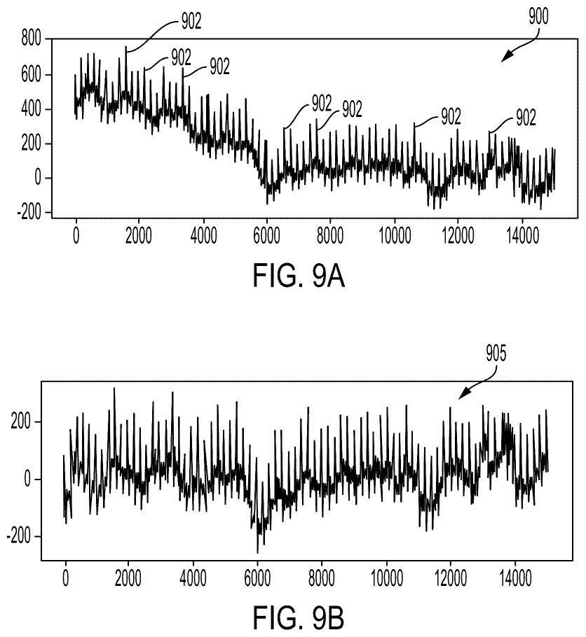

The sensor interface 304 can include physiological signal circuitry that is coupled to one or more externally applied sensors 320. The externally applied sensors 320 may include, for example, one or more externally applied physiological sensors. As shown, the sensors may be coupled to the medical device controller 300 via a wired or wireless connection. The externally applied sensors 320 may include the ECG electrodes 202 configured to sense one or more electrical signals indicative of ECG activity from the skin surface of the patient 100, as well as one or more non-ECG sensors such as a cardiovibration sensor 322 and a tissue fluid monitor 324 (e.g., configured similarly to the thoracic fluid sensor implemented through at least one RF antenna and RF circuitry discussed in further detail below with reference to FIG. 8). In implementations, the sensor interface 304 and/or the processor 318 may be configured to provide digitized ECG signals of the patient 100 based on electrical signals sensed by the ECG electrodes 202. In this sense, the sensor interface 304 and/or the processor 318 may be considered an ECG digitizing circuit. The digitized ECG signals of the patient 100 may be stored in the data storage 306.

In implementations, the cardiac event detector 314 can be configured to monitor the patient's ECG signal for an occurrence of a cardiac event such as an arrhythmia or other similar cardiac event. The cardiac event detector 314 can be configured to operate in concert with the processor 318 to execute one or more methods that process received ECG signals from, for example, the ECG sensing electrodes 202 and determine the likelihood that the patient 100 is experiencing a cardiac event, such as an arrhythmia that the device 118 is configured to treat. The cardiac event detector 314 can be implemented using hardware or a combination of hardware and software. For instance, in some examples, the cardiac event detector 314 can be implemented as a software component that is stored within the data storage 306 and executed by the processor 318. In this example, the instructions included in the cardiac event detector 314 can cause the processor 318 to perform one or more methods for analyzing a received ECG signal to determine whether an adverse cardiac event is occurring, such as an arrhythmia that the device 118 is configured to treat. In other examples, the cardiac event detector 314 can be an application-specific integrated circuit (ASIC) that is coupled to the processor 318 and configured to monitor ECG signals for adverse cardiac event occurrences. Thus, examples of the cardiac event detector 314 are not limited to a particular hardware or software implementation.

In implementations, the wearable cardiac sensing device 102 may be configured as a different wearable device from the example embodiments illustrated in FIGS. 1-3. For instance, FIG. 4 illustrates another example of a cardiac event monitoring system for removing short-term noise artifacts from an ECG signal of a patient 100, according to implementations disclosed herein. The cardiac event monitoring system of FIG. 4 includes another embodiment of the wearable cardiac sensing device 102 configured to be bodily-attached to the patient 100. The cardiac event monitoring system of FIG. 4 includes a wearable cardiac sensing device 102 configured for to be bodily-attached to the patient 100 (e.g., for long-term continuous wear by the patient 100), where the wearable cardiac sensing device 102 is in communication with the remote server 104. The wearable cardiac sensing device 102 includes a number of physiological sensors. As an example, the physiological sensors may include one or more ECG electrodes configured to sense electrical signals from a skin surface of the patient 100. In the example of FIG. 1, the wearable cardiac sensing device 102 includes a cardiac sensing unit 106 removably coupled to a removable adhesive patch 108.

The cardiac event monitoring system shown in FIG. 4 (including the remote server 104, technician interface(s) 114, and caregiver interface(s) 116) may generally function similarly to the cardiac event monitoring system shown and described above with respect to FIG. 1. Similar to the example wearable cardiac sensing device 102 shown in FIG. 1, the example wearable cardiac sensing device 102 shown in FIG. 4 is configured for long-term and/or extended use or wear by, or attachment or connection to, the patient 100, as discussed above. Such substantially or nearly continuous use, monitoring, or wear similar to the usages described above may nonetheless be considered continuous use, monitoring, or wear. In implementations, the example wearable cardiac sensing device 102 of FIG. 4 may include a portable gateway 110, where the portable gateway facilitates communication between the cardiac sensing unit 106 and the remote server 104. The portable gateway 110 may, additionally or alternatively, be configured to perform some or all of the analysis described below.

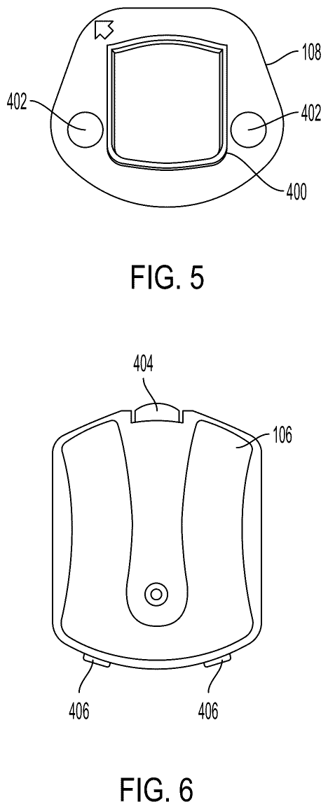

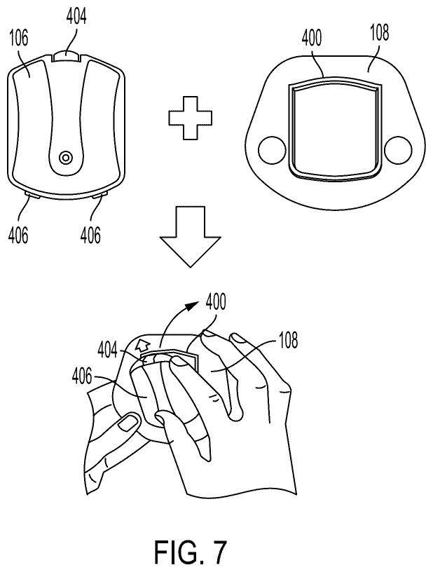

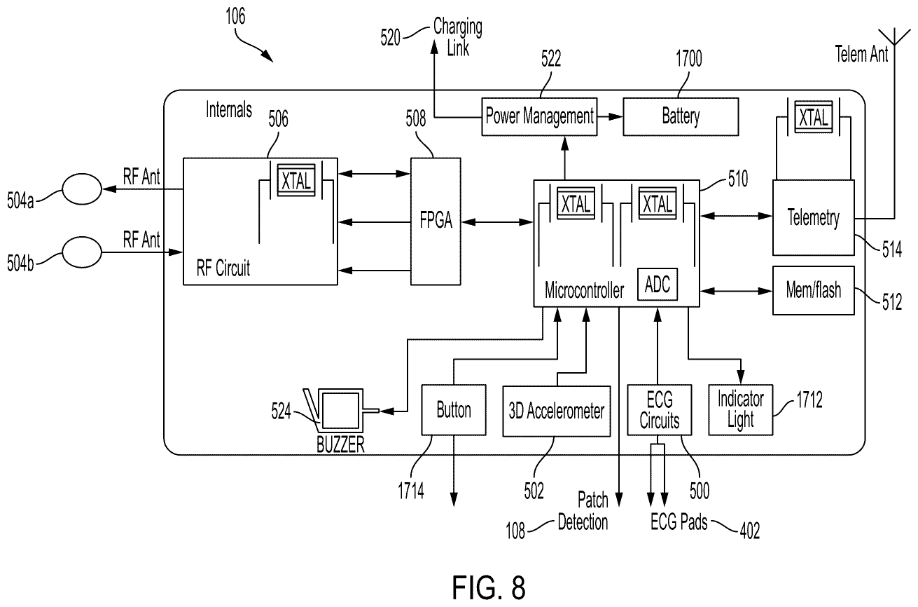

With reference to FIG. 4, the adhesive patch 108 is configured to be adhesively attached or coupled to the skin of the patient 100. The adhesive patch 108 is further configured such that the cardiac sensing unit 106 may be removably attached to the adhesive patch 108. For example, referring to FIG. 5, the adhesive patch 108 may include a frame 400 (e.g., a plastic frame) delineating the boundary of the region of the adhesive patch 108 that is configured for receiving the cardiac sensing unit 106. The adhesive patch 108 may be disposable (e.g., single-, few-, or multiple-use patches) and may be made of biocompatible, non-woven material. In some embodiments, the cardiac sensing unit 106 may be designed for long-term, continuous usage. In such embodiments, the connection between the cardiac sensing unit 106 and the adhesive patch 108 may be configured to be reversible. For example, the cardiac sensing unit 106 may be configured to be removably attached to the adhesive patch 108. In implementations, as shown in FIG. 6, the cardiac sensing unit 106 may include components such as a snap-in clip 404 that is configured to secure the cardiac sensing unit 106 to the adhesive patch 108 upon attachment to the patch frame 400. After the cardiac sensing unit 106 is attached to the frame 400, a user may press the snap-in clip 404 to subsequently release the cardiac sensing unit 106 from the frame 400. In implementations, as further shown in FIG. 6, the cardiac sensing unit 106 may include positioning tabs 406 that facilitate the attachment process between the cardiac sensing unit 106 and the adhesive patch 108. For example, the positioning tabs 406 may guide a user to insert the cardiac sensing unit 106 onto the correct portion of the frame 400 such that the cardiac sensing unit 106 can then be coupled, connected, or snapped into the frame 400 using the snap-in clip 404, as shown in FIG. 7.

Referring back to FIG. 4, FIG. 4 illustrates example locations on the body of the patient 100 where an adhesive patch 108 housing the cardiac sensing unit 106 may be placed. As an example, the adhesive patch 108 may be placed on the side of the patient's thorax (e.g., under the patient's armpit, level with a circumferential line running across the patient's chest). As another example, the adhesive patch 108 may be placed on the front of the patient's thorax (e.g., to the left of a medial line running between the patient's collarbone, for patients without dextrocardia). However, the adhesive patch 108 may also be placed on any part of the patient's thorax that allows for efficient monitoring and recording of signals from and/or associated with the patient 100. For instance, the adhesive patch 108 may be placed over the patient's sternum, over a lower portion of the patient's front or side thorax (e.g., under the patient's sternum), and/or the like.

The cardiac sensing unit 106 and adhesive patch 108 are configured for long-term and/or extended use or wear by, or attachment or connection to, the patient 100. For example, devices as described herein are capable of being continuously used or continuously worn by, or attached or connected to, the patient 100 without substantial interruption (e.g., for 24 hours, 2 days, 5 days, 7 days, 2 weeks, 30 days or 1 month, or beyond such as multiple months or even years). In some implementations, such devices may be removed for a period of time before use, wear, attachment, or connection to the patient 100 is resumed. As an illustration, the cardiac sensing unit 106 may be removed for charging, to carry out technical service, to update the device software or firmware, for the patient 100 to take a shower, and/or for other reasons or activities without departing from the scope of the examples described herein. As another illustration, the patient 100 may remove a used adhesive patch 108, as well as the cardiac sensing unit 106, so that the patient 100 may adhere a new adhesive patch 108 to their body and attach the cardiac sensing unit 106 to a new adhesive patch 108. Such substantially or nearly continuous use, monitoring, or wear as described herein may nonetheless be considered continuous use, monitoring, or wear.

For example, in implementations, the adhesive patch 108 may be designed to maintain attachment to skin of the patient 100 for several days (e.g., in a range from about 4 days to about 10 days, from about 3 days to about 5 days, from about 5 days to about 7 days, from about 7 days to about 10 days, from about 10 days to about 14 days, from about 14 days to about 30 days, etc.). After the period of use, the adhesive patch 108 can be removed from the patient's skin and the cardiac sensing unit 106 can be removed from the adhesive patch 108. The cardiac sensing unit 106 can then be removably coupled, connected, or snapped onto a new adhesive patch 108 and reapplied to the patient's skin.

As further shown in FIG. 4, the wearable cardiac sensing device 102 may include a portable gateway 110 configured to facilitate communication between the wearable cardiac sensing device 102 and the remote server 104. In implementations, the portable gateway 110 is configured to receive data and/or signals provided by the wearable cardiac sensing device 102 and transmit the data and/or signals to the remote server 104. As an illustration, in implementations, the portable gateway 110 may communicate with the remote server 104 via Ethernet, via Wi-Fi, via near-field communication (NFC), via radiofrequency, via cellular networks, via Bluetooth®-to-TCP/IP access point communication, and/or the like. Accordingly, the portable gateway 110 may include communications circuitry configured to implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In implementations, the wearable cardiac sensing device 102 and/or the portable gateway 110 may include communications circuitry that is part of an Internet of Things (IoT) and communicate with each other and/or the remote server 104 via IoT protocols (e.g., Constrained Application Protocol (CoAP), Message Queuing Telemetry Transport (MQTT), Wi-Fi, Zigbee, Bluetooth®, Extensible Messaging and Presence Protocol (XMPP), Data-Distribution Service (DDS), Advanced Messaging Queuing Protocol (AMQP), and/or Lightweight M2M (LwM2M)).

Alternatively, in other implementations, the wearable cardiac sensing device 102 may be configured to transmit data and/or signals directly to the remote server 104 instead of, or in addition to, transmitting the signals to the portable gateway 110. Accordingly, the wearable cardiac sensing device 102 may be in wired or wireless communication with the remote server 104. As an illustration, the wearable cardiac sensing device 102 may communicate with the remote server 104 via Ethernet, via Wi-Fi, via near-field communication (NFC), via radiofrequency, via cellular networks, via Bluetooth®-to-TCP/IP access point communication, and/or the like. Further, in some implementations, the cardiac event monitoring system may not include the portable gateway 110. In such implementations, the wearable cardiac sensing device 102 may perform the functions of the portable gateway 110 described herein. Additionally, in implementations where the wearable cardiac sensing device 102 is configured to communicate directly with the remote server 104, the wearable cardiac sensing device 102 may include communications circuitry configured to implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In implementations, as indicated above, the communications circuitry in the wearable cardiac sensing device 102 may be part of an IoT and communicate with the remote server 104 via IoT protocols for handling secure (e.g., encrypted) messaging and routing.

The wearable cardiac sensing system may also include a charger 112, as further shown in FIG. 4. In implementations, the charger 112 includes charging cradles configured to hold and recharge the cardiac sensing unit 106 and the portable gateway 110. Alternatively, in some implementations, the cardiac event monitoring system may not include the portable gateway 110, and accordingly, the charger 112 may be configured to hold the cardiac sensing unit 106 alone. In implementations, the cardiac sensing unit 106 and the portable gateway 110 may have separate chargers, or one or both of the cardiac sensing unit 106 and the portable gateway 110 may have removable batteries that may be replaced and/or recharged.

The remote server 104 is configured to receive and process the data and/or signals received from the wearable cardiac sensing device 102 as discussed hereinabove. The remote server 104 of FIG. 4 functions similarly to the remote server 104 of FIG. 1 as discussed hereinabove. In addition, as shown in FIG. 4, in implementations, the cardiac event monitoring system further includes one or more user interfaces, such as technician interfaces 114 and caregiver interfaces 116. The technician interfaces 114 and caregiver interfaces 116 are in electronic communication with the remote server 104 through a wired or wireless connection. The technician interfaces 114 and caregiver interfaces 116 of FIG. 4 function similarly to the technician interfaces 114 and caregiver interfaces 116 of FIG. 1 as discussed hereinabove.

As noted above, the wearable cardiac sensing device 102 includes a number of physiological sensors configured to sense signals from and/or associated with the patient 100. To illustrate, in embodiments of the wearable cardiac sensing device 102 that includes a cardiac sensing unit 106 and an adhesive patch 108, as described above with reference to FIG. 4, the physiological sensors may be implemented in the cardiac sensing unit 106 and/or implemented in the adhesive patch 108. For example, at least a portion of the physiological sensors may be integrated in the adhesive patch 108. As another example, at least a portion of the physiological sensors may be configured to be removably mounted onto the adhesive patch 108 (e.g., through being incorporated in or otherwise implemented via the cardiac sensing unit 106, which is configured to be removably mounted on to the adhesive patch 108 as discussed above).

In implementations, a number of ECG electrodes 402 may be embedded into the adhesive patch 108, as shown in FIG. 5. The ECG electrodes 402 are configured to sense one or more electrical signals indicative of ECG activity from a skin surface of the patient 100 (e.g., skin contacted directly or through a covering). The ECG electrodes 402 may be coupled to the cardiac sensing unit 106 by dedicated wiring within the adhesive patch 108, where the cardiac sensing unit 106 receives electrical signals sensed by the ECG electrodes 402 via the dedicated metal contacts or traces on the cardiac sensing unit 106 when the cardiac sensing unit 106 is mounted on the adhesive patch 108. Example ECG electrodes 402 include a metal electrode with an oxide coating, such as tantalum pentoxide electrodes. For instance, digital sensing electrodes can include skin-contacting electrode surfaces that may be deemed polarizable or non-polarizable depending on a variety of factors, including the metals and/or coatings used in constructing the electrode surface. All such electrodes can be used with the principles, techniques, devices, and systems described herein. As an illustration, the electrode surfaces can be based on stainless steel, noble metals such as platinum, or Ag—AgCl.

In examples, the ECG electrodes 402 can be used with an electrolytic gel dispersed between the electrode surface and the patient's skin. In other examples, the ECG electrodes 402 can be dry electrodes that do not need an electrolytic material or optionally can be used with an electrolytic material. For instances, such a dry electrode can be based on tantalum metal and have a tantalum pentoxide coating, as is described above. Such dry electrodes may be more comfortable for long-term monitoring applications, in various implementations.

In implementations, the ECG electrodes 402 can include additional components such as accelerometers, acoustic signal detecting devices, cardiovibrational sensors, or other measuring devices for additional parameters. For example, the ECG electrodes 402 may be configured to detect other types of physiological signals, such as thoracic fluid levels, heart vibrations, lung vibrations, respiration vibrations, patient movement, etc. Alternatively, or additionally, the cardiac sensing unit 106 and/or the adhesive patch 108 may include sensors or detectors separate from the ECG electrodes 402, such as separate motion detectors, bioacoustics sensors, cardiovibrational sensors, respiration sensors, temperature sensors, pressure sensors, and/or the like.

FIG. 8 illustrates an example electronic architecture for the cardiac sensing unit 106. The ECG circuit 500 may receive signals from the ECG electrodes 402 when the cardiac sensing unit 106 is attached to the adhesive patch 108, where the signals received from the ECG electrodes 402 include ECG waveforms sensed from the patient 100. In embodiments, the ECG electrodes 402 and cardiac sensing unit 106 may have a sampling rate in a range from about 250 Hz to about 500 Hz, from about 300 Hz to about 450 Hz, from about 350 Hz to about 400 Hz, including values and subranges therebetween. In embodiments, the cardiac sensing unit 106 may sample the signals from the ECG electrodes 402 after applying band-pass filtering by a 12 bit ADC. In implementations, during normal operation, the cardiac sensing unit 106 may transfer data from the ECG electrodes 402 to the remote server 104 “as-is,” where the remote server 104 uses the data for analysis as described in further detail below. Alternatively, in other implementations, the cardiac sensing unit 106 may perform at least some processing of the data from the ECG electrodes 402 (e.g., filtering of the data, analysis of the data, etc.). For example, the cardiac sensing unit 106 may generated digitized ECG signals from the data received from the ECG electrodes 402 and transmit the digitized ECG signals to the remote server 104 (e.g., directly or via the portable gateway 110).

In implementations, the wearable cardiac sensing device 102 can be designed to include a digital front-end where analog signals sensed by skin-contacting electrode surfaces of a set of digital sensing electrodes are converted to digital signals for processing. In this respect, the ECG circuit 500 may be an ECG digitizing circuit configured to provide digitized ECG signals of the patient 100 based on the electrical signals sensed from the patient 100. Typical wearable, ambulatory medical devices with analog front-end configurations use circuitry to accommodate a signal from a high source impedance from the sensing electrode (e.g., having an internal impedance range from approximately 100 Kiloohms to one or more Megaohms). This high source impedance signal is processed and transmitted to a monitoring device (e.g., the microcontroller 510 discussed below) for further processing. In certain implementations, the monitoring device, or another similar processor such as a microprocessor or another dedicated processor operably coupled to the sensing electrodes, can be configured to receive a common noise signal from each of the sensing electrodes, sum the common noise signals, invert the summed common noise signals and feed the inverted signal back into the patient as a driven ground using, for example, a driven right leg circuit to cancel out common mode signals.

In examples, the wearable cardiac sensing device 102 includes a processor operationally coupled to, for example, the ECG circuit 500. In embodiments, the processor may be implemented as a microcontroller 510, as shown in FIG. 5. The microcontroller 510 stores instructions specifying how measurements (e.g., ECG, accelerometer, RF, etc.) are taken, how obtained data are transmitted, how to relay a status of the cardiac sensing unit 106, how/when the cardiac sensing unit 106 can enter a sleep level, and/or the like. In implementations, the instructions may specify the conditions for performing certain types of measurements. For example, the instructions may specify that a sensor of and/or connected to the cardiac sensing unit 106 may not commence measurements (e.g., ECG measurements, radiofrequency (RF) measurements, respiration rate measurements, and/or the like) unless the patient 100 is at rest or maintaining a certain posture. As another example, the instructions may identify the conditions that have to be fulfilled before any measurements can commence, such as a sufficient attachment level between the cardiac sensing unit 106 and the adhesive patch 108, or a sufficient attachment level between the adhesive patch 108 and the surface of the patient's body to which the adhesive patch 108 is attached. In implementations, the microcontroller 510 may have internal and/or external non-volatile memory banks (e.g., memory 512) that can be used for measurement directories and data, scheduler information, and/or a log of actions and errors. This non-volatile memory may allow for retaining data and status information in the case of a total power down. In implementations, instead of a microcontroller 510 as described above, the cardiac sensing unit 106 may include a microprocessor connected to a separate non-volatile memory, such as the memory 512.

The memory 512 may also be configured as a non-transitory memory configured to store data and/or signals of the cardiac sensing unit 106. For instance, the memory 512 may be configured to store digitized ECG signals of the patient 100.

The cardiac sensing unit 106 may further be able to establish wireless communications channels with other devices, such as the portable gateway 110 and/or the remote server 104, using a telemetry or wireless communications circuit 514. For example, the wireless communications circuit 514 may be a Bluetooth® unit. Additionally, or alternatively, the wireless communications circuit 514 may include other modules facilitating other types of wireless communication (e.g., Wi-Fi, cellular, etc.). The cardiac sensing unit 106 may transmit data to the remote server 104 using the wireless communications circuit 514. In implementations, the cardiac sensing unit 106 may transmit data indirectly to the remote server 104, such as by transmitting the signals and/or data to the portable gateway 110, with the portable gateway 110 transmitting the data received from the cardiac sensing unit 106 to the remote server 104. In implementations, the cardiac sensing unit 106 may instead transmit the data directly to the remote server 104.

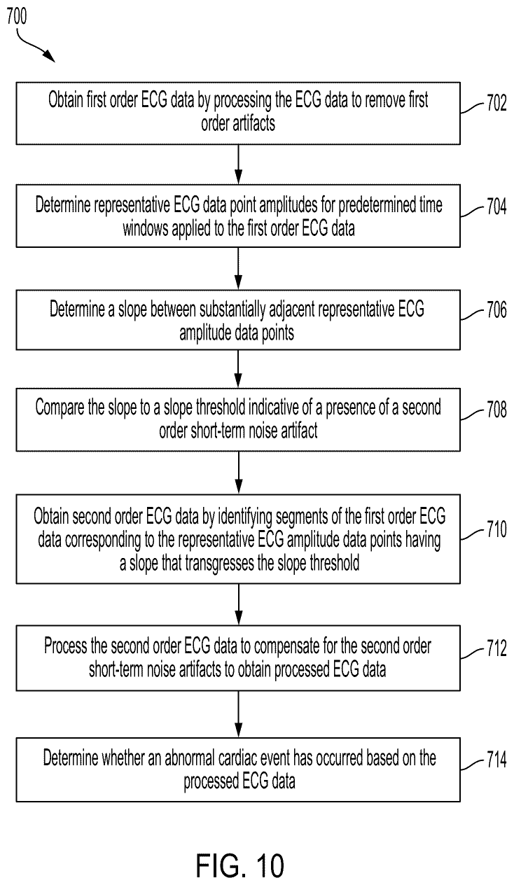

Regardless of the embodiment of the wearable cardiac sensing device 102 used, a processor in communication with the wearable cardiac sensing device 102 may be configured to process ECG data gathered by the wearable cardiac sensing device 102 to eliminate short-term noise artifacts from the ECG data and determine abnormal cardiac events in the patient 100. With reference to FIG. 9A, the raw ECG data obtained by the ECG electrodes of the wearable cardiac sensing device may include noise in the form of both first order artifacts and second order artifacts. In some examples, first order artifact filtering can be based on a finite impulse response (FIR) filter that is configured to filter the ECG signals in a range from around 0.1-50 Hz, 1-25 Hz, or 10-20 Hz based on implementation design. In some examples, first order artifact filtering can be based on a full spectrum filtering (e.g., the entire range of frequencies inherent in the ECG signals). In some examples, the first order filtering is performed based on a low pass filter to eliminate predetermined high frequency components. For example, a median filter can be used to remove baseline wander and low frequency components, and a notch filter can be used at, e.g., 50-60 Hz to eliminate AC signal interference. In an example shown in FIG. 9A, an ECG snippet 900 is shown. The vertical axis is graduated in terms of nominal amplitude and/or magnitude of the ECG signal, while the horizontal axis is graduated in terms of a sample bin index, in this case, each unit of the index corresponds to 4 milliseconds of the ECG signal. The spikes 902 are QRS complexes of each of the patient's heart beat. ECG snippet 900 is the ECG data prior to first and second order filtering. As shown, there is significant low frequency baseline wander in the ECG snippet 900. Specifically, the ECG snippet 900 baseline drifts from around 600-400 on the vertical axis to around 0 on the vertical axis.

Referring now to FIG. 9B, there is shown an ECG snippet 905 that is output after first order filtering as described herein. For example, such first order filtering includes processing the ECG snippet 900 of FIG. 9A through an FIR filter that is set at a range of around 0.05-40 Hz. As shown, in ECG snippet 905, the filter performed baseline removal by centering the strip at 0 amplitude and effectively flattened the wandering baseline.

In examples, first order artifacts can be caused by one or more of baseline wander (as discussed above), electrostatic interference, electromagnetic interference, and/or triboelectric interference lasting for durations of longer than 10 seconds or more, e.g., 30 seconds or longer, 1 minute or longer, 1 hour or longer, and/or a continuously present interference effect in the underlying ECG data. Prior to the second order signal processing described hereinafter, these first order artifacts are removed from the ECG signal. Typically, this is done utilizing various filtering techniques as described above. For example, an FIR filter may be utilized to remove noise or artifacts from the ECG data. In addition or alternatively, a frequency-based Fast Fourier Transform (FFT) detector that applies a full spectrum filtering may be utilized. In other implementations, a low-pass filter may be utilized to eliminate high frequency components from the ECG data, a median filter may be applied for baseline removal by centering the ECG data at zero amplitude and flattening the wandering baseline and to eliminate low frequency components, and a notch filter may be provided to eliminate AC interference. The results of this signal processing yields ECG data with the first order artifacts removed, which is referred to herein as first order ECG data.

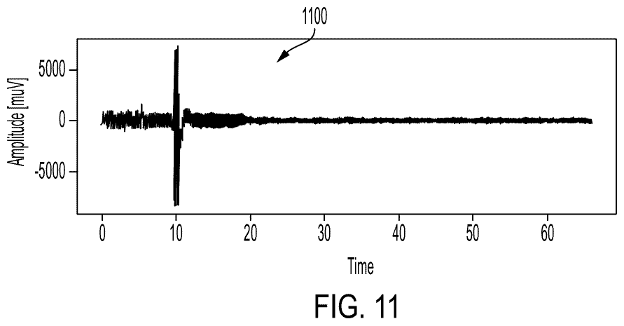

Turning now to second order filtering, such second order artifacts may be caused by short-term events such as motion by the patient causing a brief shifting of the ECG electrodes, short term electrostatic interference, short term electromagnetic interference, and/or short term triboelectric interference. For example, a short term event may have a duration of at least one of about 0.1 ms, 10 ms, 100 ms, 1 second, 2 seconds, 5 seconds, or 10 seconds. With reference to FIG. 10, the devices, methods, and techniques discussed herein processes the first order ECG data for these short term noise artifacts and provide second order ECG data. The second order ECG data may then be used by a cardiac event monitor provided on the wearable cardiac sensing device 102, the portable gateway 110, and/or the server 104 to provide improved ECG signal analysis to determine abnormal cardiac conditions in a patient because a clearer ECG data is utilized in the abnormal cardiac condition determination.

The processing discussed with regard to FIGS. 9A and 9B can be implemented by a processor in communication with the wearable cardiac sensing device 102.

FIG. 10 illustrates a sample process flow for removing short-term noise artifacts from an electrocardiogram (ECG) signal. The sample process 700 shown in FIG. 10 can be implemented by a processor in communication with the wearable cardiac sensing device 102. In implementations, the processor may be part of the wearable cardiac sensing device 102. For example, where the wearable cardiac sensing device 102 includes a cardiac sensing unit 106 and a portable gateway 110, the processor may be implemented by the microcontroller 510 of the cardiac sensing unit 106. As another example, the processor may be implemented in the portable gateway 110. As another example, where the wearable cardiac sensing device 102 includes a garment-based sensing device 118, the processor may be implemented by the processor 318 (e.g., in conjunction with the cardiac event detector 314). As another example, the processor may be implemented in the remote server 104. As another example, the processor may be implemented partially by a processor at the wearable cardiac sensing device 102 and partially by a processor at the remote server 104.

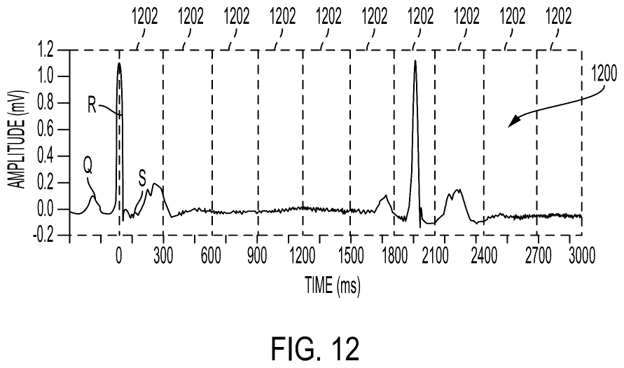

The processor is configured to first receive the ECG data obtained from the patient 100 by the ECG electrodes from memory. An example of such ECG data is illustrated in FIG. 9A. The memory may be, for example, where the wearable cardiac sensing device 102 includes a cardiac sensing unit 106 and a portable gateway 110, the memory 312 of the cardiac sensing unit 106. As another example, the processor may be implemented in the portable gateway 110. As another example, where the wearable cardiac sensing device 102 includes a garment-based sensing device 118, the memory may be implemented by the data storage 206. As another example, the memory may be implemented in the server 104. In yet another example, the processor may receive the ECG data from the ECG electrodes. The processor is further configured to obtain first order ECG data by processing the ECG data to remove first order artifacts at step 702. In implementations, as described above, the processor removes first order artifacts from the ECG signal utilizing various filtering techniques. The results of this signal processing yields ECG data with the first order artifacts removed, which is referred to herein as first order ECG data. FIG. 11 illustrates such first order ECG data 1100. The first order ECG data may be obtained by the wearable cardiac sensing device 102, such as at the cardiac sensing unit 106, at the portable gateway 110, or at the controller 300 of the garment-based sensing device 118). Alternatively, the first order ECG data may be obtained at a processor of the server 104.

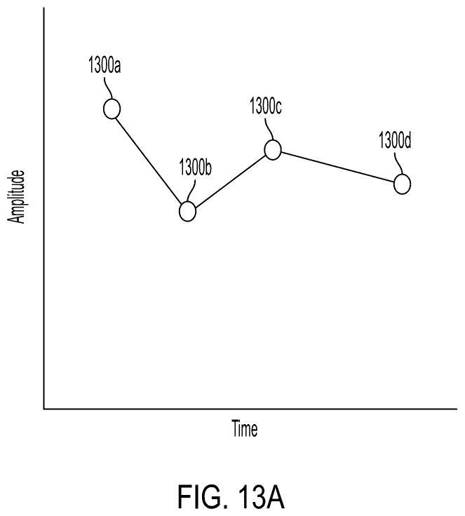

The processor may determine representative ECG data point amplitudes for predetermined time windows applied to the first order ECG data at step 704. With reference to FIG. 12, exemplary first order ECG data 1200 is illustrated. In order to determine the representative ECG data point amplitudes, predetermined time windows 1202 are applied to the first order ECG data 1200. As shown in FIG. 12, the predetermined time windows may have a duration of 300 ms. However, this is not to be construed as limiting the present disclosure as the predetermined time windows 1202 may have any suitable duration between about 1 ms and 1000 ms. For example, the predetermined time windows 1202 may have a duration of about 1 ms, about 10 ms, about 100 ms, or about 1000 ms. Once the predetermined time windows 1202 are applied to the first order ECG data 1200, the representative ECG data point amplitudes are determined. In one implementation, the representative ECG data point amplitudes are determined by averaging the amplitudes of the ECG data within the predetermined time window and assigning an average value as the representative ECG data point amplitude. In another implementation, an ensemble averaging technique may be utilized in which the representative ECG data point amplitude is determined by aligning the group of ECG signals within the predetermined time window which occur at the same time as a landmark for a given cardiac cycle. For example, the time window can be based on a 1 ms window around an R peak or about the P wave.

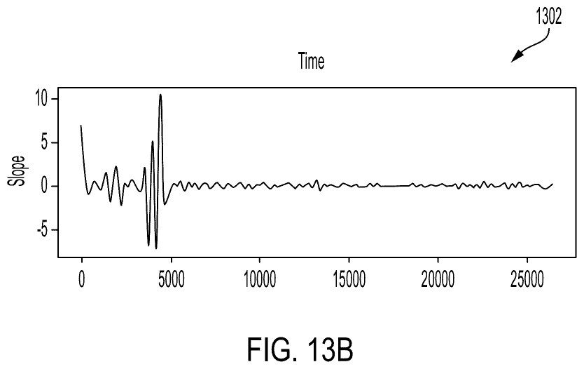

With reference to FIG. 13A, a graph illustrating the representative ECG amplitude data points 1300a, 1300b, 1300c, 1300d is illustrated. The processor thereafter determines a slope between substantially adjacent representative ECG amplitude data points 1300a, 1300b, 1300c, 1300d at step 706. Substantially adjacent representative ECG amplitude data points may be representative ECG amplitude data points that are adjacent in time order. The slope of a line connecting substantially adjacent representative ECG amplitude data points, for example points 1300a and 1300b, is determined. In one implementation, these slopes are determined using a linear equation y=mx+b, where m is the slope, for each line connecting two substantially adjacent representative ECG amplitude data points. In other implementations, a cubic spline interpolation utilizing the following equation is fit to all of the representative ECG amplitude data points.

$${S}_{i} ( x ) = {a}_{i} + {b}_{i} \cdot ( x - {x}_{i} ) + {c}_{i} \cdot {( x - {x}_{i} )}^{2} + {d}_{i} \cdot {( x - {x}_{i} )}^{3}$$

In such an implementation, the first term coefficient bi is utilized as the slope value. FIG. 13B provides a graph 1302 of the determined slopes over time for the first order ECG data 1100 of FIG. 11.

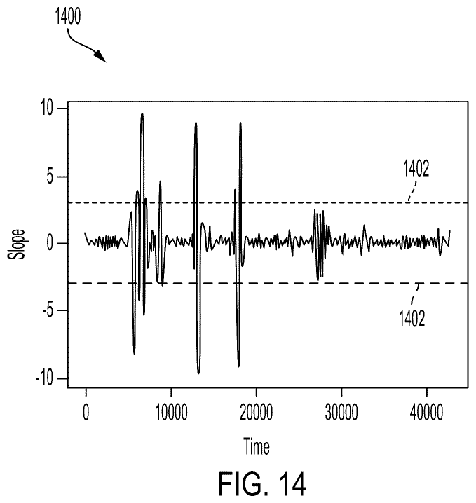

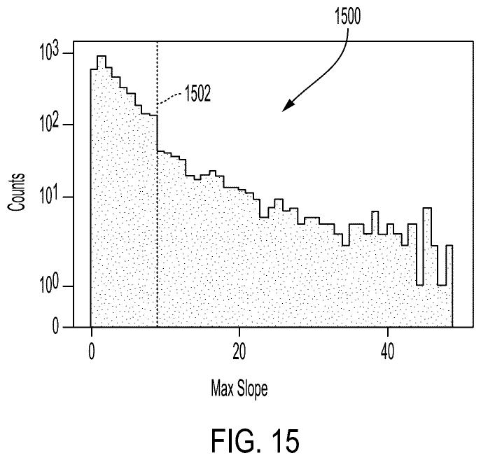

The processor then compares the slope to a slope threshold indicative of a presence of a second order short-term noise artifact at step 708. As discussed above, a second order short-term noise artifact is typically introduced into the ECG data by short term events such as motion by the patient. This motion causes a brief shifting of the ECG electrodes, which introduces noise into the ECG data. Second order short-term motion artifacts may also be introduced into the ECG data by, for example, electrostatic interference, electromagnetic interference, and/or triboelectric interference. A short term event may have a duration of at least one of about 0.1 ms, 10 ms, 100 ms, 1 second, 2 seconds, 5 seconds, or 10 seconds. With reference to FIG. 15, the slope threshold is determined by examining a distribution of the slopes determined in step 706 over a large sample of the ECG data. In examples, a minimum large sample size would be about 60 seconds of ECG data. Generally, longer samples provide better results. As such, in implementations, depending on design choices and the sizing of other parameters, the sample size can be 120 seconds of ECG data, 180 seconds of ECG data, 260 seconds of ECG data, 360 seconds of ECG data, 580 seconds of ECG data, or 1000 seconds of ECG data. An example of a distribution of the slopes 1500 is illustrated in FIG. 15. As can be seen in FIG. 15, there is a transition in the distribution denoted by the dashed line 1502. This transition 1502 denotes the slope threshold. In this example and as shown in FIG. 14, the slope threshold is set at 3, but it may also be 2, 5, 7, or any suitable value based on the distribution of the slopes. Once the slope threshold is set, the slope values determined in step 706 are compared to the slope threshold. As can be seen from FIGS. 13A and 13B, the slope values that are generated may be both positive and negative. Accordingly, the comparison of step 708 is based on the absolute value of the determined slopes. FIG. 14 illustrates a graph 1400 of the determined slopes over time with the slope threshold denoted by the dashed lines 1402.

Thereafter, the processor obtains second order ECG data (shown in FIG. 9C) by identifying segments of the first order ECG data (FIG. 9B) corresponding to the representative ECG amplitude data points having a slope that transgresses the slope threshold at step 710. For example, with reference to FIG. 14, the representative ECG amplitude data points having a slope that transgresses the slope threshold indicated by the dashed lines 1402 is indicated as having second order noise artifacts.

The processor then processes the second order ECG data to compensate for the second order short-term noise artifacts to obtain processed ECG data at step 712. In one implementation, the second order short-term noise artifacts are compensated for by removing the segments of the first order ECG data corresponding to the representative ECG amplitude data points having a slope that transgresses the slope threshold. In another implementation, the second order ECG data is processed by averaging segments of the ECG data that neighbor the first order ECG data corresponding to the representative ECG amplitude data points having a slope that transgresses the slope threshold. For example, the next 10 to 100 segments of the neighboring ECG data may be averaged. Once the average is obtained, the first order ECG data corresponding to the representative ECG amplitude data points having a slope that transgresses the slope threshold can be replaced with the average to obtain the processed ECG data. In yet another implementation, the second order ECG data is processed by determining a noise score for the first order ECG data corresponding to the representative ECG amplitude data points having a slope that transgresses the slope threshold. Methods for calculating a noise score can include measuring the signal-to-noise ratio, or measuring the variance of the amplitude.

In some implementations, the processor then determines whether an abnormal cardiac event has occurred based on the processed ECG data at step 714. For example, the cardiac event detector 314 (see FIG. 3) of the garment-based sensing device 118 can be configured to monitor the processed ECG data to determine an occurrence of a cardiac event such as an arrhythmia or other similar cardiac event. The cardiac event detector 314 can be configured to operate in concert with the processor to execute one or more methods that process the processed ECG data to determine the likelihood that the patient 100 is experiencing a cardiac event, such as a treatable arrhythmia. As discussed above, the cardiac event detector 314 can be implemented using hardware or a combination of hardware and software. For instance, in some examples, the cardiac event detector 314 can be implemented as a software component that is executed by the processor. In such an example, the instructions included in the cardiac event detector 314 can cause the processor to perform one or more methods for analyzing the processed ECG data to determine whether an adverse cardiac event is occurring, such as an arrhythmia. The analysis of such second order ECG data provides improved ECG signal analysis to determine abnormal cardiac conditions in a patient because a clearer ECG data is utilized in the abnormal cardiac condition determination.

While the process of FIG. 7 was discussed with regard one channel of ECG data, as discussed above, multiple channels of ECG data may be utilized. For example, and with reference to FIG. 2, a first ECG channel may be associated with a first electrode 202 positioned proximate a front of the patient 100 and a second electrode 202 positioned proximate a back of the patient (e.g., a front-to-back (FB) channel) and a second ECG channel may be associated with a third electrode 202 positioned proximate a first side of the patient and a fourth electrode 202 positioned proximate a second side of the patient (e.g., a side-to-side (SS) channel). Accordingly, if the process of FIG. 7 was performed on the ECG data obtained from the FB channel (i.e., a primary ECG channel), then the SS channel (i.e., a secondary ECG channel) may be utilized by the processor to validate the presence of the second order short-term noise artifact.

In addition, in certain implementations, the process 700 may be performed by a classifier stored in a non-transitory computer readable medium (e.g., a memory, a programmable circuit board, a field programmable gate array, an integrated circuit, any combination thereof, and/or the like) and implemented by the processor. The classifier may include at least one cognitive neural network trained based on a historical collection of ECG signal portions with known short-term noise artifacts. In some implementations, a neural network model is trained using ECG signals labelled as noisy. The output of the model can be binary (noise/no-noise), or it can also include a noise output along with identification of other rhythm patterns (normal sinus, atrial fibrillation, etc.).

In implementations, and as discussed above, the processor performing sample process 700 may be at the remote server 104 or may be at the wearable cardiac sensing device 102. For example, the wearable cardiac sensing device 102 may perform all of the sample process 700 and transmit an indication of an abnormal cardiac event to the remote server 104. In implementations, the wearable cardiac sensing device 102 may be configured to perform part of sample process 600 and transmit data to the remote server 104 to complete the sample process 700. As an example, the wearable cardiac sensing device 102 may identify and transmit the processed ECG data to the remote server 104 for processing.

Returning to the wearable cardiac sensing device 102, in implementations and as discussed above, the wearable cardiac sensing device 102 may include and/or be connected to one or more non-ECG physiological sensors. To illustrate, the wearable cardiac sensing device 102 may include or be connected to a motion sensor (e.g., an accelerometer) configured to sense motions of the patient 100, a cardiovibration sensor configured to sense cardiovibrations of the patient 100, a respiration sensor configured to sense respirations of the patient 100, a thoracic fluid sensor configured to sense thoracic fluid levels in the patient 100, a blood pressure sensor configured to sense a blood pressure of the patient 100, and/or the like. Other examples of non-ECG physiological sensors may include a P-wave sensor (e.g., a sensor configured to monitor and isolate P-waves within an ECG waveform), an oxygen saturation sensor (e.g., implemented through photoplethysmography, such as through light sources and light sensors configured to transmit light into the patient's body and receive transmitted and/or reflected light containing information about the patient's oxygen saturation), and so on.

As an illustration, with respect to the wearable cardiac sensing device 102 shown and described above with respect to FIGS. 4-8, the cardiac sensing unit 106 may include and/or be connected to one or more motion sensors. As shown in FIG. 8, the cardiac sensing unit 106 may include, for example, a 3D accelerometer 502 with three axes and a span of +2 g. Using the 3D accelerometer 502, the cardiac sensing unit 106 may acquire data on the patient's movements and activity level, data on the patient's posture (e.g., angle of posture), data on cardiovibrations of the patient's heart (e.g., vibrations corresponding to the opening and closing of the patient's heart valves), data on the patient's respirations (e.g., data on the patient's respiration rate), and/or the like. In implementations, the cardiac sensing unit 106 may include a different type of motion sensor, such as a 1-axis channel accelerometer, 2-axis channel accelerometer, gyroscope, magnetometer, or ballistocardiography sensor.

As another example, the cardiac sensing unit 106 may include an RF sensor configured to take bio-impedance measurements of the patient's thorax. In implementations, the cardiac sensing unit 106 may then transmit the bio-impedance measurements to the remote server 104, which uses the bio-impedance measurements to determine a thoracic fluid level in the patient 100. In implementations, the cardiac sensing unit 106 may determine a thoracic fluid level in the patient 100 from the bio-impedance measurements and transmit the thoracic fluid level to the remote server 104. Accordingly, as shown in FIG. 8, an example embodiment of the cardiac sensing unit 106 includes at least one RF antenna, such as a transmitting RF antenna 504a and a receiving RF antenna 504b (or a single antenna configured to transmit and receive RF waves, in other implementations), and RF circuitry 506 configured to transmit a low-power signal in an ultra-high frequency band (e.g., 0.1 GHz to 5.0 GHz, 0.5 GHz to 2.1 GHZ) at a predetermined rate (e.g., every 10 ms, every 20 ms, every 30 ms, every 40 ms, every 50 ms, etc.). The cardiac sensing unit 106 may also include other circuits for controlling the RF circuitry 506 (e.g., field-programmable gate array (FPGA) circuits 508). The at least one RF antenna 504a, 504b and RF circuitry 506 receive RF-based biosignals indicative of the thoracic fluid level in the patient 100 in the form of RF waves transmitted through the patient 100 and/or scattered or reflected from the patient 100. For example, the at least one RF antenna 504a, 504b and RF circuitry 506 may detect transmitted, scattered, and/or reflected RF waves for a predetermined amount of time (e.g., about 30 seconds, about 1 minute, about 2 minutes, about 3 minutes, about 5 minutes, about 10 minutes, etc.).

In implementations, the wearable cardiac sensing device 102 (e.g., at the cardiac sensing unit 106 and/or at the portable gateway 110) and/or the remote server 104 are configured to gate when RF measurements are taken and/or discard certain RF measurements based on the patient's state when the RF measurements were taken. For example, the wearable cardiac sensing device 102 and/or the remote server 104 may determine whether the patient 100 showed movement above a predetermined threshold before the wearable cardiac sensing device 102 started the RF measurements process and/or while the RF was taking place. If RF measurements were taken during or immediately after movement above the predetermined threshold, the remote server 104 may discard those RF measurements.

In implementations, the wearable cardiac sensing device 102 shown and described with respect to FIGS. 4-8 may include and/or be connected to other types of non-ECG physiological sensors from the sensors shown in FIG. 5, in addition to the sensors discussed above and/or in the alternative to the sensors discussed above. For example, the wearable cardiac sensing device 102 may include and/or be connected to a bioacoustics sensor, such as a microphone, configured to detect cardiovibrations and/or cardiac acoustics from the patient 100 (e.g., instead of an accelerometer). In implementations, the wearable cardiac sensing device 102 may include and/or be connected to an impedance-based transthoracic sensor configured to sense transthoracic signals indicative of respiration of the patient 100 (e.g., instead of an accelerometer).

In implementations, the wearable cardiac sensing device 102 may include and/or be connected to a blood pressure sensor, such as a blood pressure sensor implemented through an RF sensor (e.g., including the at least one RF antenna 504a, 504b and RF circuitry 506 discussed above) combined with a photoplethysmography (PPG) sensor (e.g., including one or more light emitting diodes (LEDs)). For example, the wearable cardiac sensing device 102 may use the RF sensor to generate information about an aortic waveform (e.g., a waveform of aortic volume over time) and the PPG sensor to generate information about an arterial waveform for arteries a known distance from the aorta. As an illustration, the RF sensor may be provided in the cardiac sensing unit 106 as discussed above, where the cardiac sensing unit 106 and the adhesive patch 108 are located over the patient's sternum. The cardiac sensing unit 106 may transmit RF waves into the patient's thorax and receive reflected and/or scattered RF waves, which the wearable cardiac sensing device 102 uses to generate information about the patient's aortic waveform (e.g., at the cardiac sensing unit 106 and/or the portable gateway 110). The PPG sensor may be provided in the cardiac sensing unit 106 and/or in the adhesive patch 108 and configured to transmit light waves into and receive scattered and/or reflected light waves from surface arteries over the patient's sternum. The wearable cardiac sensing device 102 then uses the received light waves to generate information about the patient's arterial waveform (e.g., at the cardiac sensing unit 106 and/or the portable gateway 110). As another illustration, instead of the cardiac sensing unit 106 and/or adhesive patch 108 including the PPG sensor, the PPG sensor may be provided in another device configured with the PPG sensor, such as a wristband or armband. The device with the PPG sensor may be electronically and/or communicably coupled to the wearable cardiac sensing device 102 and/or to the portable gateway 110.

Once the information about the aortic waveform and the arterial waveform has been generated, the wearable cardiac sensing device 102 may determine the patient's pulse wave velocity by identifying a fiducial point on the aortic waveform (e.g., a point of highest volume or a point of onset in the aorta in a cardiac cycle), identifying a corresponding fiducial point on the arterial waveform (e.g., a point of highest volume or a point of onset in the surface arteries in the same cardiac cycle), determine a time difference between the two fiducial points, and divide the known distance between the aorta and the surface arteries being measured by the time difference between the two fiducial points. The wearable cardiac sensing device 102 may then determine the patient's blood pressure from the pulse wave velocity. Alternatively, the wearable cardiac sensing device 102 may transmit the information about the patient's aortic waveform and the information about the patient's arterial waveform to the remote server 104. The remote server 104 may then perform some or all of this analysis. Additional details on implementations of a blood pressure sensor as described above may be found in U.S. patent application Ser. No. 17/656,480, filed on Mar. 25, 2022, titled “SYSTEM FOR USING RADIOFREQUENCY AND LIGHT TO DETERMINE PULSE WAVE VELOCITY,” which is hereby incorporated by reference. Other examples of sensors configured to sense additional types of signals that may be incorporated into the wearable cardiac sensing device 102 shown and described with respect to FIGS. 4-8 may include temperature sensors, pressure sensors, humidity sensors, and/or the like.

In implementations, the distribution of the externally-applied biosignal sensors of the wearable cardiac sensing device 102 shown and described with respect to FIGS. 4-8 may be different from the examples discussed above. For instance, the ECG pads 402 may be provided on the cardiac sensing unit 106 instead of on the adhesive patch 108. The adhesive patch 108 may accordingly include an aperture or another opening whereby the ECG pads 402 of the cardiac sensing unit 106 may contact the skin of the patient 100. As another illustration, the adhesive patch 108 may include one or more motion sensors, such as the 3D accelerometer 502.

In implementations, the patient 100 may instruct the wearable cardiac sensing device 102 shown and described with respect to FIGS. 4-8 to record symptom events. For example, in embodiments, the patient 100 may provide a symptom input via the portable gateway 110 of the wearable cardiac sensing device 102. As an illustration, the portable gateway 110 may include a visual display and a processor for the portable gateway 110 in communication with the visual display. As such, the portable gateway 110 may be configured to display, on the visual display, a screen that includes a list of potential cardiac-related symptoms to the patient 100. Example symptoms may include lightheadedness, a racing heart, fatigue, fainting, a fall, chest discomfort, a skipped heartbeat, and shortness of breath. The portable gateway 110 further may be configured to receive a selection by the patient 100 of one or more of the displayed potential cardiac-related symptoms. For instance, the visual display may include a touchscreen, where the patient 100 interacts with the touchscreen to select the cardiac-related symptom(s) the patient 100 is experiencing. As another example, the patient 100 may use an input device of the portable gateway 110, such as arrow buttons, a tracking pad, or a keyboard, to select the cardiac-related symptom(s) that the patient 100 is experiencing. The portable gateway 110 thus records the selected cardiac-related symptom(s) and the time the patient 100 submitted the cardiac-related symptom(s).

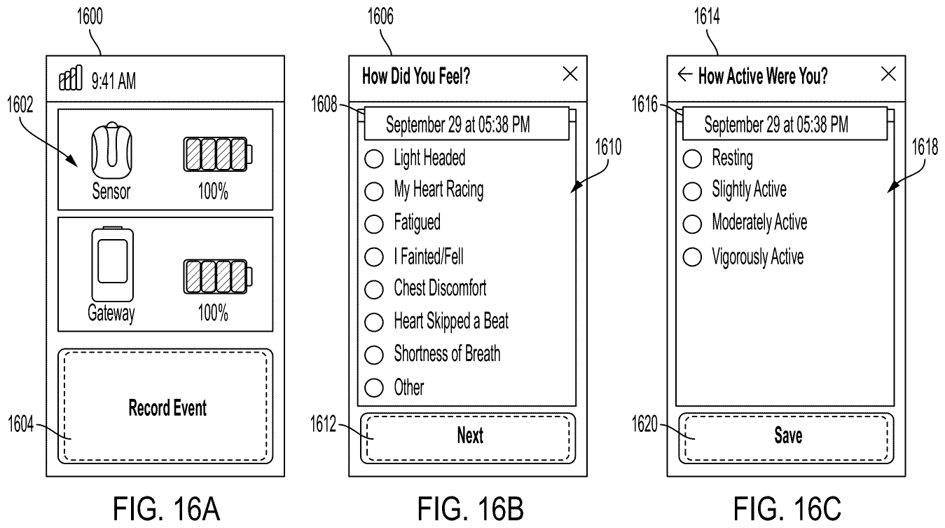

To illustrate, FIGS. 16A-16C show example screens that may be displayed on a visual display of the portable gateway 110 and used by the patient 100 to provide the symptom input. FIG. 16A shows an example home screen 1600 that may be displayed by the portable gateway 110, for instance, as a default screen for the portable gateway 110. The home screen 1600 may include information on the wearable cardiac sensing device 102 such as battery level indicators 1602 (e.g., for the portable gateway 110 and the cardiac sensing unit 106). Additionally, the home screen 1600 includes a “Record Event” button 1604 that the patient 100 can press if the patient 100 is experiencing a suspected cardiac-related symptom.

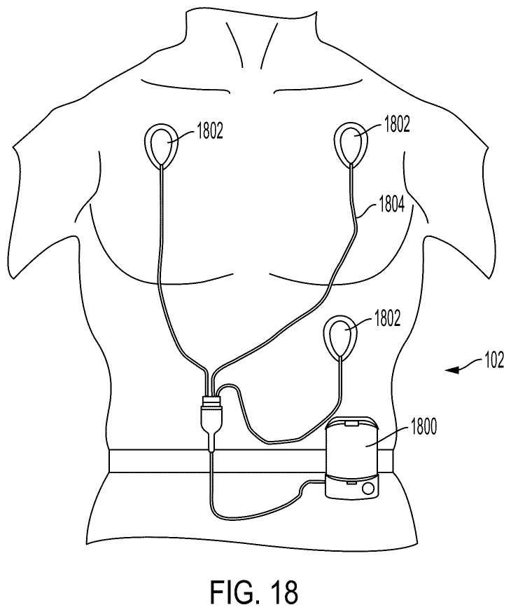

In response to the patient 100 selecting the “Record Event” button 1604, the portable gateway 110 may display a symptom screen 1606, an example of which is shown in FIG. 16B. The symptom screen may include a subsection 1608 listing the time and date that the patient 100 is reporting the suspected cardiac-related symptom (e.g., the time and date the patient 100 selected the “Record Event” button 1604). In addition, the symptom screen 1606 includes a list 1610 of potential cardiac-related symptoms. As an illustration, in the example of FIG. 18B, the list 1610 of potential cardiac-related symptoms may include Light-Headed,” “My Heart Racing,” “Fatigued,” “I Fainted/Fell,” “Chest Discomfort,” “Heart Skipped a Beat,” “Shortness of Breath,” and “Other” (e.g., in response to the selection of which the patient 100 is shown a screen onto which the patient 100 may type their “other” symptom(s)). The symptom screen 1606 may also include a “Next” button 1612 that the patient 100 can press once the patient 100 has selected the suspected cardiac-related symptom(s) from the list 1610.

In response to the patient 100 selecting the “Next” button 1612, the portable gateway 110 may display an activity screen 1614, an example of which is shown in FIG. 16C. The activity screen 1614 may include a subsection 1616 that again lists the time and date that the patient 100 is reporting the suspected cardiac-related symptom. The activity screen 1614 also includes a list 1618 of types of potential activities that the patient 100 may have been engaging in when the patient 100 started experiencing the recorded symptom(s). As shown in the example of FIG. 16C, the list 1618 of potential types of activity may include “Resting,” “Slightly Active,” “Moderately Active,” and “Vigorously Active.” The activity screen 1614 may also include a “Next” button 1620 that the patient 100 can press once the patient 100 has selected the type of activity the patient 100 was experiencing from the list 1618. Once the patient 100 has selected the “Next” button 1620, the portable gateway 110 may transmit the selected cardiac-related symptom(s) from the list 1610 of potential cardiac-related symptoms, the selected type of activity from the list 1618 of potential types of activity, and the time and date of the symptom input.

In implementations, the example screens described above may be displayed on a user interface disposed on the wearable cardiac sensing device 102, e.g., on a housing of the cardiac sensing unit 106. The patient 100 may thus provide the symptom input via the cardiac sensing unit 106. For example, the front of the cardiac sensing unit 106 (e.g., the face of the cardiac sensing unit 106 facing away from the patient 100 when the cardiac sensing unit 106 is being worn by the patient 100) may be configured to receive a patient input. As an illustration, the front of the cardiac sensing unit 106 may include a button that the patient 100 can tap, or the cardiac sensing unit 106 may sense changes in electrical charge on the front surface by the patient 100 tapping on the front surface. The patient 100 can then provide a symptom input to the cardiac sensing unit 106 via the front face of the cardiac sensing unit 106. An example process for providing a symptom input to the cardiac sensing unit 106 may include (1) the patient 100 remaining still, (2) double tapping the front face of the cardiac sensing unit 106 with the palm of the patient's hand, and (3) waiting for a beeping sound from the cardiac sensing unit 106, which indicates that the symptom input has been recorded. If the beeping sound does not occur, the patient 100 may need to re-tap the cardiac sensing unit 106 to record the symptom input.

Furthermore, in addition to recording the symptom input from the patient 100, the wearable cardiac sensing device 102 is configured to record one or more biosignal segments associated with the symptom input. In implementations, the wearable cardiac sensing device 102 is configured to record the one or more biosignal segments within a predetermined time period before and after the symptom input. For example, the wearable cardiac sensing device 102 may record the one or more biosignal segments 30 seconds, 1 minute, 2 minutes, 3 minutes, 4 minutes, 5 minutes, etc. before and/or after the symptom input. In some cases, the wearable cardiac sensing device 102 may record the one or more biosignal segments for a predetermined time period after the symptom input. In some cases, the wearable cardiac sensing device 102 may record the one or more biosignal segments for a first predetermined time period before the symptom input and a second predetermined time period after the symptom input. The first predetermined time period may be the same as the second predetermined time period, or the first predetermined time period may be different from the second predetermined time period. As an illustration, the wearable cardiac sensing device 102 may record the one or more biosignal segments for 30 seconds before the symptom input and 1 minute after the symptom input. The biosignal(s) for the one or more biosignal segments may include any of the biosignal(s) discussed above, such as ECG signals, cardiovibrational signals, respiration signals, thoracic fluid signals, and/or the like. In implementations, the wearable cardiac sensing device 102 may also record one or more additional signal segments. For example, the wearable cardiac sensing device 102 may record accelerometer signal segments containing activity level and posture information for the patient 100. The wearable cardiac sensing device 102 may be configured to transmit data associated with the symptom event (e.g., including an indication of the symptom event and/or the one or more biosignal segments recorded in association with the symptom event) to the remote server 104.

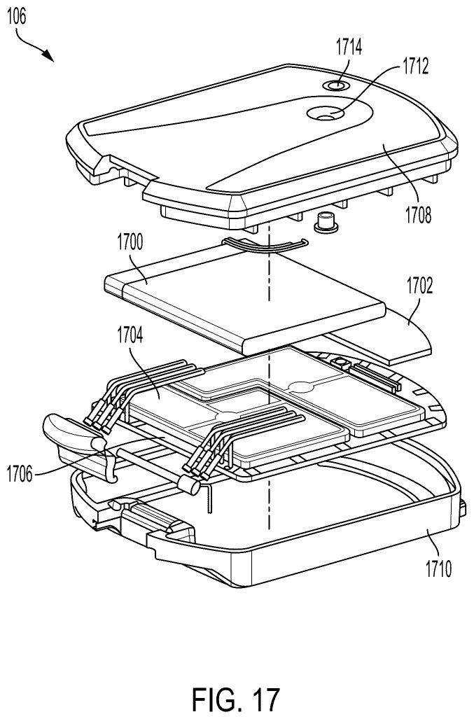

In implementations, the wearable cardiac sensing device 102 of FIGS. 4-8 may also include additional components to those described above. FIG. 17 provides an exploded view of the cardiac sensing unit 106, according to some implementations. The exploded view of FIG. 17 illustrates various components of the cardiac sensing unit 106. For example, the cardiac sensing unit 106 may include a power source, such as a battery 1700. In examples, the battery 1700 may be a rechargeable lithium ion battery configured to supply power for at least one month of continuous or near-continuous recording of the one or more biosignals. The cardiac sensing unit 106 may also include a wireless communications circuit 1702, a radiofrequency shield 1704 (e.g., a metallic cover, for instance, to prevent interference with RF processing and other digital circuitry), a digital circuit board 1706, and/or the like. The wireless communications circuit 1702 may be a Bluetooth® unit, in some implementations, although in addition to or alternatively to the Bluetooth® unit, other modules facilitating other types of communications (e.g., Wi-Fi, cellular, etc.) may be included in the cardiac sensing unit 106.

In implementations, the cardiac sensing unit 106 may be configured to monitor, record, and transmit data or signals (e.g., the biosignal-based data) to the portable gateway 110 continuously (e.g., via the wireless communications circuit 1702). The cardiac sensing unit 106 monitoring and/or recording additional data may not interrupt the transmission of already acquired data to the portable gateway 110. As such, in implementations, both the monitoring/recording and the transmission processes may occur at the same time or nearly the same time. In implementations, if the cardiac sensing unit 106 does suspend the monitoring and/or recording of additional data while the cardiac sensing unit 106 is transmitting already acquired data to the portable gateway 110, the cardiac sensing unit 106 may then resume monitoring and/or recording of additional data before all of the already-acquired data has been transmitted to the portable gateway 110. To illustrate, the interruption period for the monitoring and/or recording of additional data may be less in comparison to the time it takes the cardiac sensing unit 106 to transmit the already-acquired data (e.g., the interruption period being between about 0% to 80%, about 0% to about 60%, about 0% to about 40%, about 0% to about 20%, about 0% to about 10%, about 0% to about 5%, including values and subranges therebetween, of the monitoring and/or recording period). This moderate interruption period may facilitate the near-continuous monitoring and/or recording of additional data during transmission of already-acquired physiological data. For example, in one scenario, when a measurement time is about two minutes, any period of suspension or interruption in the monitoring and/or recording of subsequent measurement data may range from a few milliseconds to about a minute. Illustrative reasons for such suspension or interruption of data may include allowing for the completion of certain data integrity and/or other online tests of previously acquired data. In some implementations, if the previous data have problems, the cardiac sensing unit 106 may notify the patient and/or a remote technician of the problems so that appropriate adjustments can be made.

In implementations, the cardiac sensing unit 106 may be configured to monitor, record, and transmit some data in a continuous or near-continuous manner, as discussed above, while monitoring, recording, and transmitting some other data in a non-continuous manner (e.g., periodically, non-periodically, etc.). As an illustration, the cardiac sensing unit 106 may be configured to record and transmit ECG data from the ECG electrodes 402 continuously or nearly continuously while data from at least one RF antenna and RF circuitry (e.g., the at least one RF antenna 504a, 504b and the RF circuitry 506) is transmitted periodically. For example, RF measurements may be taken only when the patient 100 is in a good position for transmitting and receiving RF waves, such as when the patient 100 is not moving. As such, biosignal-based data including ECG data may be transmitted to the portable gateway 110 (and, via the portable gateway 110, to the remote server 104) continuously or near-continuously as additional ECG data is being recorded, while biosignal-based data including RF data may be transmitted once the RF measuring process is completed. In implementations, monitoring and/or recording of signals by the cardiac sensing unit 106 may be periodic and, in some implementations, may be accomplished as scheduled (e.g., periodically) without delay or latency during the transmission of already acquired data to the portable gateway 110. For example, the cardiac sensing unit 106 may take measurements from the patient 100 and transmit the data generated from the measurements to the portable gateway 110 in a continuous manner as described above.

Additionally, in implementations, the portable gateway 110 may continuously transmit the signals provided by the cardiac sensing unit 106 to the remote server 104. Thus, for example, the portable gateway 110 may transmit the signals from the cardiac sensing unit 106 to the remote server 104 with little or no delay or latency. To this end, in the context of data transmission between the wearable cardiac sensing device 102 and the remote server 104, continuously includes continuous (e.g., without interruption) or near continuous (e.g., within one minute after completion of a measurement and/or an occurrence of an event on the cardiac sensing unit 106). Continuity may also be achieved by repetitive successive bursts of transmission (e.g., high-speed transmission). Similarly, immediate data transmission may include data transmission occurring or done immediately or nearly immediately (e.g., within one minute after the completion of a measurement and/or an occurrence of an event on the cardiac sensing unit 106).

Further, in the context of signal acquisition and transmission by the wearable cardiac sensing device 102, continuously may also include uninterrupted collection of data sensed by the cardiac sensing unit 106 with clinical continuity. In this case, short interruptions in data acquisition of up to one second several times an hour, or longer interruptions of a few minutes several times a day, may be tolerated and still seen as continuous. As to latency as a result of such a continuous scheme as described herein, the overall amount of response time (e.g., time from when an event onset is detected to when a notification regarding the event is issued) can amount, for example, from about five to fifteen minutes. As such, transmission/acquisition latency may therefore be in the order of minutes.

In implementations, the bandwidth of the link between the cardiac sensing unit 106 and the portable gateway 110 may be larger, and in some instances, significantly larger, than the bandwidth of the acquired data to be transmitted via the link (e.g., burst transmissions). Such implementations may ameliorate issues that may arise during link interruptions, periods of reduced/absent reception, etc. In implementations, when transmission is resumed after an interruption, the resumption may be in the form of last-in-first-out (LIFO). In some implementations, the portable gateway 110 additionally may be configured to operate in a store and forward mode, where the data received from the cardiac sensing unit 106 is first stored in an onboard memory of the portable gateway 110 and then forwarded to the remote server 104. In some implementations, the portable gateway 110 may function as a pipeline and pass through data from the cardiac sensing unit 106 immediately to the remote server 104. Further, in implementations, the data from the cardiac sensing unit 106 may be compressed using data compression techniques to reduce memory requirements as well as transmission times and power consumption. In some implementations, the link between the portable gateway 110 and the remote server 104 may similarly be larger, and in some instances, significantly larger, than the bandwidth of the data to be transmitted via the portable gateway 110 and the remote server 104.

Alternatively, the wearable cardiac sensing device 102 including the cardiac sensing unit 106 may not include the portable gateway 110. As such, the cardiac sensing unit 106 may continuously transmit data or signals directly to the remote server 104 using similar processes as those discussed above. Additionally, in implementations, the wearable cardiac sensing device 102 may include a garment-based sensing device 118 that may or may not be in communication with a portable gateway 110. Similar processes may thus also be applied to the garment-based sensing device 118 and/or the portable gateway 110 for the purpose of continuously transmitting data or signals to the remote server 104.

Referring back to FIG. 17, the components of the cardiac sensing unit 106 (e.g., the battery 1700, communications circuit 1702, radiofrequency shield 1704, digital circuit board 1706, and/or the like) may be provided between a front cover 1708 forming an upper surface of the cardiac sensing unit 106 and a back cover 1710 forming a bottom surface of the cardiac sensing unit 106. For instance, the back cover 1710 may be configured to contact the adhesive patch 108, and the front cover 1708 may be configured to face away from the patient 100 such that the front cover 1708 is accessible when the cardiac sensing unit 106 is attached to the adhesive patch 108. In some implementations, an indicator light 1712 and/or a button 1714 may be embedded into the front cover 1708 visible through the upper surface. The indicator light 1712 may provide feedback on the status of the cardiac sensing unit 106 and its components, such as the charging and/or power level of the power source of the cardiac sensing unit 106 (e.g., the battery 1700), the attachment level of the cardiac sensing unit 106 to the adhesive patch 108, the attachment level of the adhesive patch 108 to the surface of the body to which the adhesive patch 108 is attached, etc.

The button 1714 may be configured for the patient 100 and/or a caregiver of the patient 100 to provide feedback to the cardiac sensing unit 106 and/or, via the cardiac sensing unit 106, to the remote server 104. For instance, the button 1614 may allow the patient 100 and/or caregiver to activate or deactivate the cardiac sensing unit 106. In some implementations, the button 1714 may be used to reset the cardiac sensing unit 106, as well as pair the cardiac sensing unit 106 to the portable gateway 110 and initiate communication with the portable gateway 110. In some implementations, the button 1714 may allow a user to set the cardiac sensing unit 106 in an “airplane mode,” for example, by deactivating any wireless communication (e.g., Wi-Fi, Bluetooth®, etc.) with external devices and/or servers, such as the portable gateway 110 and/or the remote server 104.

Referring back to FIG. 8, the example cardiac sensing unit 106 includes additional components to those discussed above with reference to FIG. 17. In implementations, as shown in FIG. 5, the cardiac sensing unit 106 includes one or more external interfaces, either connected to or embedded in the cardiac sensing unit 106. For example, the cardiac sensing unit 106 may include the button or switch 1714 for activating the cardiac sensing unit 106, deactivating the cardiac sensing unit 106, pairing the cardiac sensing unit 106 with the portable gateway 110, receiving a patient input related to a symptom, and/or the like. In implementations, the cardiac sensing unit 106 may also include the indicator light 1712 and a buzzer 524 for providing light and/or audio feedback to a user of the cardiac sensing unit 106 (e.g., in response to the patient 100 activating the button 1714 or tapping the cardiac sensing unit 106 to record that the patient 100 is experiencing symptoms suspected to be related to an arrhythmia).

Further, in some embodiments, the cardiac sensing unit 106 may be connectable to the ECG pads or electrodes 402 coupled to the patient 100 (e.g., connectable to the ECG pads 402 embedded in the adhesive patch 108) and to a charger, such as charger 112, via a charging link 520. For instance, the back cover 1710 of the cardiac sensing unit 106 may implement the charging link 520 via metal contacts configured to connect to the ECG pads 402 when the cardiac sensing unit 106 is attached to the adhesive patch 108 and to a charging power source when the cardiac sensing unit 106 is attached to the charger 112. As discussed above, the ECG circuits 500 may then receive signals from the ECG pads 402 when the cardiac sensing unit 106 is attached to the adhesive patch 108. Alternatively or additionally, in implementations the charging link 520 may be implemented through an inductive circuit configured to charge the cardiac sensing unit 106 via a wireless inductive charging. As shown in FIG. 8, the charging link 520 may be coupled to a power management circuit 522 (e.g., when the cardiac sensing unit 106 is attached to the charger 112, when the cardiac sensing unit 106 is placed in proximity to an inductive charging pad), where the power management circuit 522 is configured to charge an onboard power source such as the battery 1700.

In implementations, at least some of the functionality described above as occurring on the cardiac sensing unit 106 may occur on the portable gateway 110. As an example, at least some of the components or processes described above as being located at and/or performed by the cardiac sensing unit 106 may be located at and/or performed by the portable gateway 110. Thus, the controller of the wearable cardiac sensing device 102 may be implemented through a combination of the cardiac sensing unit 106 and the portable gateway 110.

Returning to the wearable cardiac sensing device 102 shown and described with respect to FIGS. 1-3, any of the externally applied sensors described above as being used or incorporated into the wearable cardiac sensing device 102 of FIGS. 4-8 may also be applied to the wearable cardiac sensing device 102 of FIGS. 1-3. For instance, other examples of externally applied sensors 320 may include a respiration sensor, a bioacoustics sensor, a blood pressure sensor, a temperature sensor, a pressure sensor, a humidity sensor, a P-wave sensor (e.g., a sensor configured to monitor and isolate P-waves within an ECG waveform), an oxygen saturation sensor (e.g., implemented through photoplethysmography, such as through light sources and light sensors configured to transmit light into the patient's body and receive transmitted and/or reflected light containing information about the patient's oxygen saturation), and so on.

As an illustration, the one or more cardiovibration sensors 322 can be configured to detect cardiac or pulmonary vibration information. The one or more cardiovibration sensors 322 can transmit information descriptive of the cardiovibrations (and other types of sensed vibrations) to the sensor interface 504 for subsequent analysis. For example, the one or more cardiovibration sensors 322 can detect the patient's heart valve vibration information (e.g., from opening and closing during cardiac cycles). As a further example, the one or more cardiovibration sensors 322 can be configured to detect cardiovibrational signal values including one or more of S1, S2, S3, and S4 cardiovibrational biomarkers. From these cardiovibrational signal values or heart vibration values, certain heart vibration metrics may be calculated (e.g., at the garment-based sensing device 118 and/or at the remote server 104). These heart vibration metrics may include one or more of electromechanical activation time (EMAT), average EMAT, percentage of EMAT (% EMAT), systolic dysfunction index (SDI), or left ventricular systolic time (LVST). The one or more cardiovibration sensors 322 can also be configured to detect heart wall motion, for instance, by placement of the sensor in the region of the apical beat. In implementations, the one or more cardiovibration sensors 322 can include vibrational sensor configured to detect vibrations from the patient's cardiac and pulmonary system and provide an output signal responsive to the detected vibrations of a targeted organ. For example, the one or more cardiovibration sensors 322 may be configured to detect vibrations generated in the trachea or lungs due to the flow of air during breathing. In implementations, additional physiological information can be determined from pulmonary-vibrational signals such as, for example, lung vibration characteristics based on sounds produced within the lungs (e.g., stridor, crackle, etc.). In implementations, the one or more cardiovibration sensors 322 can include a multi-channel accelerometer, for example, a three-channel accelerometer configured to sense movement in each of three orthogonal axes such that patient movement/body position can be detected and correlated to detected cardiovibrations information.

As another illustration, in implementations, the sensor interface 304 may be connected to one or more motion sensors (e.g., one or more accelerometers, gyroscopes, magnetometers, ballistocardiographs, etc.) as part of the externally applied sensors 320. In implementations, the medical device controller 300 may include a motion detector interface, either implemented separately or as part of the sensor interface 304. For instance, as shown in FIG. 3, the medical device controller 300 may include a motion sensor interface 326 operatively coupled to one or more motion detectors 328 configured to generate motion data, for example, indicative of physical activity performed by the patient 100 and/or physiological information internal to the patient 100. Examples of a motion detector may include a 1-axis channel accelerometer, 2-axis channel accelerometer, 3-axis channel accelerometer, multi-axis channel accelerometer, gyroscope, magnetometer, ballistocardiograph, and the like. For instance, the motion data may include accelerometer counts indicative of physical activity performed by the patient 100, accelerometer counts indicative of respiration rate of the patient 100, accelerometer counts indicative of posture information for the patient 100, accelerometer counts indicative of cardiovibrational information for the patient 100, and/or the like.

The motion sensor interface 326 is configured to receive one or more outputs from the motion sensors 328. The motion sensor interface 326 can be further configured to condition the output signals by, for example, converting analog signals to digital signals (if using an analog motion sensor), filtering the output signals, combining the output signals into a combined directional signal (e.g., combining each x-axis signal into a composite x-axis signal, combining each y-axis signal into a composite y-axis signal, and combining each z-axis signal into a composite z-axis signal). In examples, the motion sensor interface 326 can be configured to filter the signals using a high-pass or band-pass filter to isolate the acceleration of the patient due to movement from the component of the acceleration due to gravity. Additionally, the motion sensor interface 326 can configure the outputs from the motion sensor 328 for further processing. For example, the motion sensor interface 326 can be configured to arrange the output of an individual motion sensor 328 as a vector expressing acceleration components of the x axis, the y-axis, and the z-axis of the motion sensor 328. The motion sensor interface 326 can thus be operably coupled to the processor 318 and configured to transfer the output and/or processed motion signals from the motion sensors 328 to the processor 318 for further processing and analysis.

In implementations, the one or more motion sensors 328 can be integrated into one or more components of the garment-based sensing device 118, either within the medical device controller 300 or external to the medical device controller 300 as shown in FIG. 3. For instance, in some implementations, the one or more motion detectors 328 may be located in or near the ECG electrodes 202. In some implementations, the one or more motion detectors 328 may be located elsewhere on the garment-based sensing device 118. For example, a motion detector 328 may be integrated into the medical device controller 300 (e.g., such that the one or more motion detectors 328 would be located within the housing 302 of the medical device controller 300, as shown in FIG. 8). In some implementations, a motion detector 328 may be integrated into another component of the garment-based sensing device 118, such as a therapy electrode 204, the connection pod 208, and/or the like. In some implementations, a motion detector 328 can be integrated into an adhesive ECG sensing and/or therapy electrode patch.

As described above, the sensor interface 304 and the motion sensor interface 326 can be coupled to any one or combination of external sensors to receive patient data indicative of patient parameters. Once data from the sensors has been received by the sensor interface 304 and/or the motion sensor interface 326, the data can be directed by the processor 318 to an appropriate component within the medical device controller 300. For example, ECG signals collected by the ECG sensors 202 may arrive at the sensor interface 304, and the sensor interface 304 may transmit the ECG signals to the processor 318, which, in turn, relays the patient's ECG data to the cardiac event detector 314. The sensor data can also be stored in the data storage 306 and/or transmitted to the remote server 104 via the network interface 308.

Further, referring back to embodiments of the wearable cardiac sensing device 102 including the garment-based sensing device 118, FIG. 3 includes additional components of the medical device controller 300 to those discussed above. In implementations, the user interface 310 may include one or more physical interface devices, such as input devices, output devices, and combination input/output devices, and a software stack configured to drive operation of the devices. These user interface elements may render visual, audio, and/or tactile content. Thus, the user interface 310 may receive inputs and/or provide outputs, thereby enabling a user to interact with the medical device controller 300.

The medical device controller 300 can also include at least one battery 312 configured to provide power to one or more components integrated in the medical device controller 300. The battery 312 can include a rechargeable multi-cell battery pack. In one example implementation, the battery 312 can include three or more cells (e.g., 2200 mA lithium ion cells) that provide electrical power to the other device components within the medical device controller 300. For example, the battery 312 can provide its power output in a range of between 20 mA to 1000 mA (e.g., 40 mA) output and can support 24 hours, 48 hours, 72 hours, or more, of runtime between charges. In certain implementations, the battery capacity, runtime, and type (e.g., lithium ion, nickel-cadmium, or nickel-metal hydride) can be changed to best fit the specific application of the medical device controller 300.

Additionally, the garment-based sensing device 118 shown in FIGS. 1-3 may be configured, in various implementations, to provide therapeutic shocks to the patient 100 upon detecting that the patient 100 is experiencing a treatable arrhythmia. The alarm manager 316 can be implemented using hardware or a combination of hardware and software. For instance, in some examples, the alarm manager 316 can be implemented as a software component that is stored within the data storage 306 and executed by the processor 318. In this example, the instructions included in the alarm manager 516 can cause the processor 518 to configure alarm profiles and notify intended recipients using the alarm profiles. In other examples, the alarm manager 316 can be an application-specific integrated circuit (ASIC) that is coupled to the processor 318 and configured to manage alarm profiles and notify intended recipients using alarms specified within the alarm profiles. Thus, examples of the alarm manager 316 are not limited to a particular hardware or software implementation.

The therapy delivery circuit 330 can be coupled to the therapy electrodes 204 configured to provide therapy to the patient 100. For example, the therapy delivery circuit 330 can include, or be operably connected to, circuitry components that are configured to generate and provide an electrical therapeutic shock. The circuitry components can include, for example, resistors, capacitors, relays and/or switches, electrical bridges such as an H-bridge (e.g., including a plurality of insulated gate bipolar transistors or IGBTs), voltage and/or current measuring components, and other similar circuitry components arranged and connected such that the circuitry components work in concert with the therapy delivery circuit 330 and under the control of one or more processors (e.g., processor 318) to provide, for example, one or more pacing, defibrillation, or cardioversion therapeutic pulses. In implementations, pacing pulses can be used to treat cardiac arrhythmias such as bradycardia (e.g., less than 30 beats per minute) and tachycardia (e.g., more than 150 beats per minute) using, for example, fixed rate pacing, demand pacing, anti-tachycardia pacing, and the like. Defibrillation or cardioversion pulses can be used to treat ventricular tachycardia and/or ventricular fibrillation.

In implementations, the therapy delivery circuit 330 includes a first high-voltage circuit connecting a first pair of the therapy electrodes 204 and a second high-voltage circuit connecting a second pair of the therapy electrodes 204 such that the first biphasic therapeutic pulse is delivered via the first high-voltage circuit and the second biphasic therapeutic pulse is delivered via the second high-voltage circuit. In implementations, the second high-voltage circuit is configured to be electrically isolated from the first high-voltage circuit. In implementations, the therapy delivery circuit 330 includes a capacitor configured to be selectively connected to the first high-voltage circuit and/or the second high-voltage circuit. As such, the first high-voltage circuit may powered by the capacitor when the capacitor is selectively connected to the first high-voltage circuit, and the second high-voltage circuit may be powered by the capacitor when the capacitor is selectively connected to the second high-voltage circuit. In implementations, the therapy delivery circuit 330 includes a first capacitor electrically connected to the first high-voltage circuit and a second capacitor electrically connected to the second high-voltage circuit.

The capacitors can include a parallel-connected capacitor bank consisting of a plurality of capacitors (e.g., two, three, four, or more capacitors). In some examples, the capacitors can include a single film or electrolytic capacitor as a series connected device including a bank of the same capacitors. These capacitors can be switched into a series connection during discharge for a defibrillation pulse. For example, four capacitors of approximately 140 μF or larger, or four capacitors of approximately 650 μF can be used. The capacitors can have a 1600 VDC or higher rating for a single capacitor, or a surge rating between approximately 350 to 500 VDC for paralleled capacitors and can be charged in approximately 15 to 30 seconds from a battery pack.

For example, each defibrillation pulse can deliver between 60 to 180 J of energy. In some implementations, the defibrillating pulse can be a biphasic truncated exponential waveform, whereby the signal can switch between a positive and a negative portion (e.g., charge directions). This type of waveform can be effective at defibrillating patients at lower energy levels when compared to other types of defibrillation pulses (e.g., such as monophasic pulses). For example, an amplitude and a width of the two phases of the energy waveform can be automatically adjusted to deliver a precise energy amount (e.g., 150 J) regardless of the patient's body impedance. The therapy delivery circuit 330 can be configured to perform the switching and pulse delivery operations, e.g., under control of the processor 318. As the energy is delivered to the patient 100, the amount of energy being delivered can be tracked. For example, the amount of energy can be kept to a predetermined constant value even as the pulse waveform is dynamically controlled based on factors, such as the patient's body impedance, while the pulse is being delivered.

In certain examples, the therapy delivery circuit 330 can be configured to deliver a set of cardioversion pulses to correct, for example, an improperly beating heart. When compared to defibrillation as described above, cardioversion typically includes a less powerful shock that is delivered at a certain frequency to mimic a heart's normal rhythm.

In response to the cardiac event detector 314 described above determining that the patient 100 is experiencing a treatable arrhythmia, the processor 318 is configured to deliver a cardioversion/defibrillation shock to the patient 100 via the therapy electrodes 204. In implementations, the alarm manager 316 can be configured to manage alarm profiles and notify one or more intended recipients of events, where an alarm profile includes a given event and the intended recipients who may have in interest in the given event. These intended recipients can include external entities, such as users (e.g., patients, physicians and other caregivers, a patient's loved one, monitoring personnel), as well as computer systems (e.g., monitoring systems or emergency response systems, which may be included in the remote server 104 or may be implemented as one or more separate systems). For example, when the processor 318 determines using data from the ECG sensing electrodes 202 that the patient 100 is experiencing a treatable arrhythmia, the alarm manager 516 may issue an alarm via the user interface 510 that the patient 100 is about to experience a defibrillating shock. The alarm may include auditory, tactile, and/or other types of alerts. In some implementations, the alerts may increase in intensity over time, such as increasing in pitch, increasing in volume, increasing in frequency, switching from a tactile alert to an auditory alert, and so on. Additionally, in some implementations, the alerts may inform the patient 100 that the patient 100 can abort the delivery of the defibrillating shock by interacting with the user interface 310. For instance, the patient 100 may be able to press a user response button or user response buttons on the user interface 310, after which the alarm manager 316 will cease issuing an alert and the medical device controller 300 will no longer prepare to deliver the defibrillating shock.

In implementations, the cardiac event detector 314 is configured to detect when the patient 100 is experiencing a cardiac rhythm change (e.g., an episode of ventricular fibrillation (VF), an episode of ventricular tachycardia (VT), a premature ventricular contraction, and/or the like) in response to a cardiac rhythm disruptive shock (e.g., coordinated by the therapy delivery circuit 330) delivered during a baselining session, as discussed above. Depending on the type of cardiac rhythm change, the processor 318 is configured to deliver a cardioversion/defibrillation shock to the patient 100 via the therapy electrodes 204, as discussed above, to restore the patient's normal cardiac rhythm. For example, if the cardiac rhythm change is VF, the processor 318 is configured to deliver a cardioversion/defibrillation shock to the patient 100.

The embodiments of the wearable cardiac sensing device 102 including the cardiac sensing unit 106, adhesive patch 108, and portable gateway 110, and the garment-based sensing device 118, described above are examples of the wearable cardiac sensing device 102. However, other embodiments of a wearable cardiac sensing device 102 may also be used with the systems and methods described herein. Examples of other embodiments of the wearable cardiac sensing device 102, for instance, are described below with respect to FIGS. 18-20.

One alternative implementation of the wearable cardiac sensing device 102 is illustrated in FIG. 18. As shown in FIG. 18, in some implementations, the wearable cardiac sensing device 102 may include a cardiac sensor unit 1800 that further includes individual electrodes 1802 configured to be adhered to the patient's body. Each individual electrode 1802 may be covered with a hydrogel for signal acquisition from the patient. The individual electrodes 1802 may be in wired communication with the sensor unit 180 via cables 1804, as shown. The sensor unit 1800 may be worn, for example, on a belt of the patient via a belt clip attachment (not shown). In implementations, the sensor unit 1800 may communicate with a portable gateway 110, which transmits signals from the sensor unit 1800 to the remote server 104, similar to implementations discussed above. In implementations, the sensor unit 1800 may communicate directly with the remote server 104, similar to implementations discussed above.

The individual electrodes 1802 may be positioned on the patient in a configuration suited for acquiring ECG signals from the patient. For example, as illustrated in FIG. 18, the wearable cardiac sensing device 102 may include three electrodes 1802, which two of the electrodes 1802 positioned near either side of the patient's collarbone and the third electrode positioned lower on the patient's thorax. Additionally, in implementations, the wearable cardiac sensing device 102 may include one or more motion sensors in the sensor unit 1800, similar to implementations of the cardiac sensing unit 106 including the adhesive patch 108 discussed above. In implementations, the wearable cardiac sensing device 102 of FIG. 18 may include one or more motion sensors connected to the individual electrodes 1802. For instance, each electrode 1802 may have a connected motion sensor, or a particular electrode 1802 may have a connected motion sensor, such as the electrode 1802 configured to be positioned lower on the patient's thorax in FIG. 18. In implementations, the wearable cardiac sensing device 102 of FIG. 18 may include one or more motion sensors connected to the cables 1804.

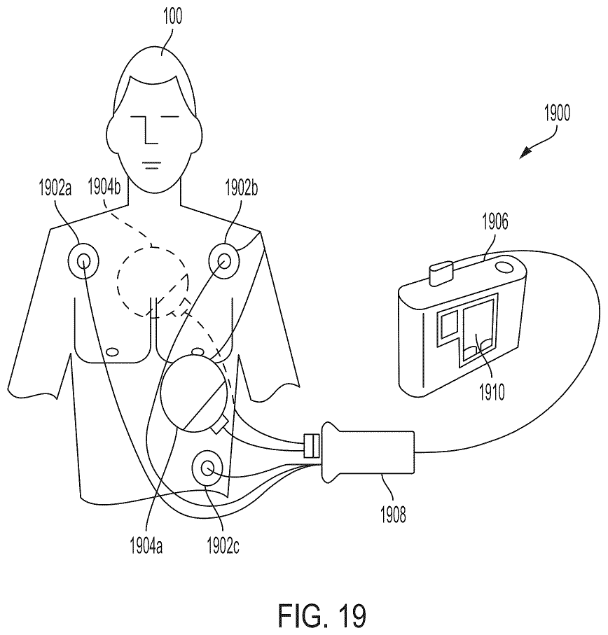

FIG. 19 illustrates another example of a wearable cardiac sensing device 102. More specifically, FIG. 19 shows a hospital wearable defibrillator 1900 that is external, ambulatory, and wearable by the patient 100. Hospital wearable defibrillator 1900 can be configured in some implementations to provide pacing therapy, e.g., to treat bradycardia, tachycardia, and asystole conditions. The hospital wearable defibrillator 1200 can include one or more ECG sensing electrodes 1902a, 1902b, 1902c (e.g., collectively ECG sensing electrodes 1902), therapy electrodes 1904a and 1904b (e.g., collectively therapy electrodes 1904), a medical device controller 1906, and a connection pod 1908. For example, each of these components can be structured and function as similar components of the embodiments of the garment-based sensing device 118 discussed above with reference to FIGS. 1-3. In implementations, the electrodes 1902 and 1904 can include disposable adhesive electrodes. For example, the electrodes 1902 and 1904 can include sensing and therapy components disposed on separate sensing and therapy electrode adhesive patches. In implementations, both sensing and therapy components can be integrated and disposed on a same electrode adhesive patch that is then attached to the patient 100.

As an illustration, the front adhesively attachable therapy electrode 1904a attaches to the front of the patient's torso to deliver pacing or defibrillating therapy. Similarly, the back adhesively attachable therapy electrode 1904b attaches to the back of the patient's torso. In an example scenario, at least three ECG adhesively attachable sensing electrodes 1902 can be attached to at least above the patient's chest near the right arm (e.g., electrode 1902a), above the patient's chest near the left arm (e.g., electrode 1902b), and towards the bottom of the patient's chest (e.g., electrode 1902c) in a manner prescribed by a trained professional. In implementations, the hospital wearable defibrillator 1900 may include additional adhesive therapy electrodes 1904 and/or the patches shown in FIG. 19 may include additional therapy electrodes 1904 on them such that at least two vectors may be formed between the therapy electrodes 1904 of the hospital wearable defibrillator 1900, as described above.

In implementations, the medical device controller 1906 may be configured to function similarly to the medical device controller 406 discussed above with respect to the garment-based sensing device 118. As shown in FIG. 21, the medical device controller 1906 may include a user interface 1910 configured to communicate information with the patient 100. In examples, the patient 100 being monitored by a hospital wearable defibrillator and/or pacing device 1900 may be confined to a hospital bed or room for a significant amount of time (e.g., 75% or more of the patient's stay in the hospital). As a result, a user interface 1910 can be configured to interact with a user other than the patient 100 (e.g., a technician, a clinician or other caregiver) for device-related functions such as initial device baselining (e.g., including performing a baselining therapy session), setting and adjusting patient parameters, and changing the device batteries.

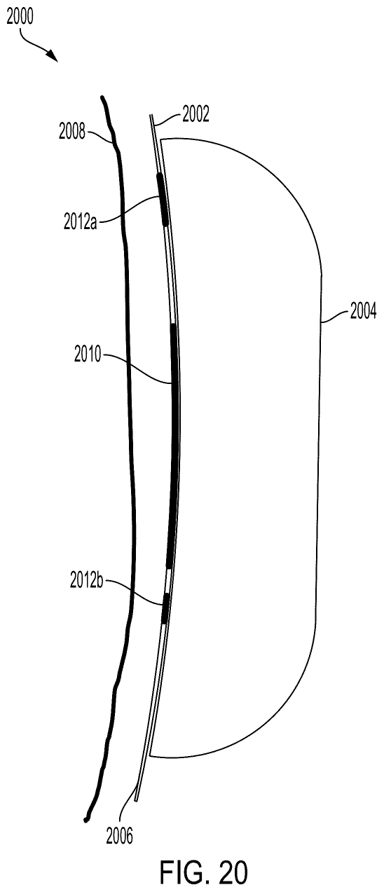

FIG. 20 illustrates another example of a wearable cardiac sensing device 102. As shown in FIG. 20, the wearable cardiac sensing device 102 may be or include an adhesive assembly 2000. The adhesive assembly 2000 includes a contoured pad 2002 and a housing 2004 configured to form a watertight seal with the contoured pad 2002. In implementations, the housing 2004 is configured to house electronic components of the adhesive assembly 2000, such as electronic components similar to components described above with respect to the cardiac sensing unit 106 of FIG. 8 and/or the medical device controller 300 of FIG. 3. The adhesive assembly 2000 includes a conductive adhesive layer 2006 configured to adhere the adhesive assembly 2000 to a skin surface 2008 of the patient 100. The adhesive layer 2006 may include, for example, a water-vapor permeable conductive adhesive material, such as a material selected from the group consisting of an electro-spun polyurethane adhesive, a polymerized microemulsion pressure sensitive adhesive, an organic conductive polymer, an organic semi-conductive conductive polymer, an organic conductive compound and a semi-conductive conductive compound, and combinations thereof.

The adhesive assembly 2000 also includes at least one of a therapy electrodes 2010 integrated with the contoured pad 2002. In implementations, the adhesive assembly 2000 may include a therapy electrode 2010 that forms a vector with another therapy electrode disposed on another adhesive assembly 2000 adhered to the patient's body and/or with a separate therapy electrode adhered to the patient's body (e.g., similar to therapy electrodes 204 shown in FIGS. 2 and 3). The adhesive assembly 2000 may also include one or more ECG sensing electrodes 2012 integrated with the contoured pad 2002 (e.g., ECG sensing electrodes 2012a and 2012b). In implementations, the adhesive assembly 2000 may alternatively or additionally be in electronic communication with a separate ECG sensing electrode, such as an adhesive sensing electrode adhered to the patient's body. In examples, as shown in FIG. 20, the therapy electrode(s) 2010 and ECG sensing electrode(s) 2012 may be formed within the contoured pad 2002 such that a skin-contacting surface of each component is coplanar with or protrudes from the patient-contacting face of the contoured pad 2002. Examples of a wearable cardiac sensing device 102 including an adhesive assembly 2000 are described in U.S. patent application Ser. No. 16/585,344, entitled “Adhesively Coupled Wearable Medical Device,” filed on Sep. 27, 2019, now U.S. Pat. No. 11,890,461, which is hereby incorporated by reference in its entirety.

Although the subject matter contained herein has been described in detail for the purpose of illustration, such detail is solely for that purpose and that the present disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

Other examples are within the scope and spirit of the description and claims. Additionally, certain functions described above can be implemented using software, hardware, firmware, hardwiring, or combinations of any of these. Features implementing functions can also be physically located at various positions, including being distributed such that portions of functions are implemented at different physical locations.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. Those skilled in the art will readily appreciate that all parameters, dimensions, materials, and configurations described herein are meant to be an example and that the actual parameters, dimensions, materials, and/or configurations will depend upon the specific application or applications for which the inventive teachings is/are used.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

1. A cardiac event monitoring system for removing short-term noise artifacts from an electrocardiogram (ECG) signal, comprising:
a wearable cardiac sensing device configured to be bodily-attached to the patient, the wearable cardiac sensing device comprising
a plurality of physiological sensors comprising one or more ECG electrodes configured to sense electrical signals from a skin surface of the patient; and
a non-transitory memory configured to store ECG data based on the electrical signals of the patient; and
a processor in communication with the wearable cardiac sensing device, the processor configured to
receive the ECG data from the memory;
obtain first order ECG data by processing the ECG data to remove first order artifacts;
determine representative ECG data point amplitudes for predetermined time windows applied to the first order ECG data;
determine a slope between substantially adjacent representative ECG amplitude data points;
compare the slope to a slope threshold indicative of a presence of a second order short-term noise artifact;
obtain second order ECG data by identifying segments of the first order ECG data corresponding to the representative ECG amplitude data points having a slope that transgresses the slope threshold;
process the second order ECG data to compensate for the second order short-term noise artifacts to obtain processed ECG data; and
determine whether an abnormal cardiac event has occurred based on the processed ECG data.

2. The cardiac event monitoring system of claim 1, wherein the second order ECG data is processed by removing the segments of the first order ECG data corresponding to the representative ECG amplitude data points having a slope that transgresses the slope threshold.

3. The cardiac event monitoring system of claim 1, wherein the second order ECG data is processed by averaging segments of the ECG data that neighbor the first order ECG data corresponding to the representative ECG amplitude data points having a slope that transgresses the slope threshold and replacing the first order ECG data corresponding to the representative ECG amplitude data points having a slope that transgresses the slope threshold with the average.

4. The cardiac event monitoring system of claim 1, wherein the second order ECG data is processed by determining a noise score for the first order ECG data corresponding to the representative ECG amplitude data points having a slope that transgresses the slope threshold.

5. The cardiac event monitoring system of claim 1, wherein the second order short-term noise artifacts comprise at least one of a motion artifact, electrostatic interference, triboelectric interference, and electromagnetic interference.

6. The cardiac event monitoring system of claim 1, wherein the processor is further configured obtain at least two ECG signal channels from the plurality of physiological sensors.

7. The cardiac event monitoring system of claim 6, wherein the at least two ECG signal channels comprise a primary ECG signal channel and a secondary ECG signal channel.

8. The cardiac event monitoring system of claim 7, wherein the secondary ECG channel is provided to validate the presence of the second order short-term noise artifact.

9. The cardiac event monitoring system of claim 1, wherein the predetermined time windows have a duration of at least one of 1 ms, 10 ms, 100 ms, or 1000 ms.

10. The cardiac event monitoring system of claim 1, wherein the short-term noise artifact is a motion artifact that lasts for a duration of at least one of about 0.1 ms, 10 ms, 100 ms, 1 second, 2 seconds, 5 seconds, or 10 seconds.

11. The cardiac event monitoring system of claim 1, wherein the slope threshold is at least one of 2, 3, 5, or 7.

12. The cardiac event monitoring system of claim 1, wherein the slope threshold is determined based on a distribution of the determined slopes of the first order ECG data.

13. The cardiac event monitoring system of claim 1, wherein the representative ECG data point amplitudes are an average of amplitudes of the first order ECG data within the predetermined time windows.

14. The cardiac event monitoring system of claim 13, wherein the average of the amplitudes of the first order ECG data is obtained using ensemble averaging.

15. The cardiac event monitoring system of claim 1, wherein the wearable cardiac sensing device further comprises a garment configured to be worn around the patient's torso for an extended period of time.

16. (canceled)

17. (canceled)

18. The cardiac event monitoring system of claim 15, wherein the plurality of physiological sensors comprise at least three physiological sensors and the garment further includes one or more therapy electrodes incorporated therein, wherein the one or more therapy electrodes are positioned within the garment to deliver one or more therapeutic defibrillating shocks to the patient.

19. The cardiac event monitoring system of claim 1, wherein the wearable cardiac sensing device further comprises a removable adhesive patch configured to be adhered to skin of the patient.

20. (canceled)

21. The cardiac event monitoring system of claim 19, wherein the wearable cardiac sensing device further comprises a cardiac sensing unit incorporating the portion of the plurality of physiological sensors, the cardiac sensing unit configured to be removably mounted onto the removable adhesive patch.

22. The cardiac event monitoring system of claim 1, wherein the wearable cardiac sensing device further comprises at least one tissue fluid monitor configured to measure a fluid level in tissue of the patient.

23. (canceled)

24. (canceled)

25. (canceled)

26. The cardiac event monitoring system of claim 1, wherein the wearable cardiac sensing device comprises the processor and is in communication with a remote server, and wherein the processor is further configured to transmit at least one of the processed ECG data or an indication of the abnormal cardiac event to the remote server.

27-78. (canceled)
